(19) United States
(12) Patent Application Publication
(10) Pub. No.: US 2008/0039522 A1
(43) Pub. Date: Feb. 14, 2008

(54) Composition Containing a Complex Comprising a Metal Ion and a Carboxylate Ligand Having Anti-Inflammatory Activity

(75) Inventors: Peter Lay, Newtown (AU); Trevor Hambley, Ashbury (AU); Jane Weder, Leichhardt (AU); Neal Davies, Pullman, WA (US); Hubertus L. Regtop, New South Wales (AU); John R. Biffen, Braemar (AU); Brendan Kennedy, Newtown (AU)

Correspondence Address: EDWARDS ANGELL PALMER & DODGE LLP, P.O. BOX 55874, BOSTON, MA, 02205, US

(73) Assignee: Medical Therapies Limited, North Ryde, New South Wales (AU)

(21) Appl. No.: 10/594,778

(22) PCT Filed: Mar. 30, 2005
(86) PCT No.: PCT/AU05/00442

§ 371 Date: Jun. 20, 2007

Related U.S. Application Data
(60) Provisional application No. 60/664,867, filed on Mar. 24, 2005.

(30) Foreign Application Priority Data
Mar. 30, 2004 (AU) 2004901694
Mar. 24, 2005 (AU) 2005901464

(51) Int. Cl. A61K 31/30; A61K 31/28; A61K 31/315; A61K 31/555; A61K 31/675; A61P 17/00; A61P 19/02; A61P 21/00; A61P 29/00

(52) U.S. Cl. 514/494; 514/184; 514/186; 514/188; 514/492; 514/499; 514/501; 514/79

(57) ABSTRACT

A pharmaceutical composition comprising a metal complex of a carboxylate having anti-inflammatory activity in a pharmaceutically acceptable carrier, wherein: (1) the composition has a colloidal structure, or forms a colloidal structure when administered to a human or animal, or is immiscible with water; (2) more than 80% of the total amount of the carboxylate having anti-inflammatory activity in the composition is present as part of a metal complex; and (3) less than 10% of the carboxylate having anti-inflammatory activity complexed with the metal dissociates from the metal over 12 months when the composition is stored in the absence of light at room temperature; but excluding compositions comprising a metal complex containing the ligand DMF. The invention also provides the use of the pharmaceutical composition in the treatment of inflammatory conditions in humans and animals.

This application claims priority from Australian provisional patent application no. 2004901694 filed on 30 Mar. 2004, Australian provisional patent application titled “Copper Complexes” filed on 24 Mar. 2005 and United States provisional patent application titled “Copper Complexes” filed on 24 Mar. 2005.

### TECHNICAL FIELD

The present invention relates to pharmaceutical compositions for the treatment of inflammatory conditions, and the use of such compositions in the treatment of inflammatory conditions in humans or animals. The compositions of the present invention contain a complex comprising a metal ion and a carboxylate ligand having anti-inflammatory activity.

### BACKGROUND ART

Non-steroidal anti-inflammatory drugs (NSAIDs) are used in the treatment of a variety of inflammatory conditions in humans and animals. NSAIDs are used to treat inflammatory conditions including rheumatoid arthritis, osteoarthritis, acute musculoskeletal disorders (such as tendonitis, sprains and strains), lower back pain (commonly referred to as lumbago), and inflammation, pain and edema following surgical or non-surgical procedures.

However, many NSAIDs cause adverse effects in humans and animals, particularly adverse gastrointestinal (GI) effects. For example, indomethacin is a NSAID and is effective in treating inflammatory conditions in humans and animals. However, indomethacin can cause severe adverse gastrointestinal effects in humans and animals, particularly when administered orally. In humans, oral administration of indomethacin can cause ulcerations in the esophagus, stomach, duodenum and intestines, and some fatalities have been reported. In dogs, oral administration of indomethacin causes fatal gastrointestinal hemorrhaging. Adverse gastrointestinal effects have also been reported for administration of indomethacin by other routes. It has also been reported that the topical administration of indomethacin causes severe adverse effects including the death of the test animals (“Anti-inflammatory activity of Indomethacin following topical application”, Amico-Roxas, M., Mater, M., Caruso, A., Puglisi, G., Bemadini, R., Rinaldo, G., European Review for Medical & Pharmacological Sciences, 1982, IV, 1999, 204). Adverse gastrointestinal effects have also been reported for administration of indomethacin by suppository. The adverse gastrointestinal effects have limited the use of many NSAIDs.

It has been found that for many NSAIDs, metal complexes of the NSAID cause less adverse side effects, and result in increased uptake of the drug, compared to the free NSAID.

For example, the oral administration of the Cu(II) complex of indomethacin, bis(N,N-dimethylformamide)tetrakis-μ-(O,O′-Indo)dicopper(II) ([Cu₂(Indo)₄(DMF)₂]), has been found to cause less adverse gastrointestinal effects than indomethacin; and it has been claimed that the complex has increased anti-inflammatory activity compared to indomethacin. The mechanism of the reduced gastrointestinal toxicity has not been elucidated. However, it is believed that it is at least in part due to the complex being more lipophilic than indomethacin, which leads to more optimal absorption of the complex.

Compositions containing the complex [Cu₂(Indo)₄(DMF)₂] sold under the name Cu-Algesic have been used in veterinary practice in Australia, New Zealand, South Africa and other countries. These compositions are in the form of a tablet or a paste. The Cu-Algesic tablets comprise 2 mg of the complex and the excipients dextrose (24.8%), cellulose (35%), maize starch (25.6%), magnesium stearate (4.27%), Tixosil (a silica based flow enhancing agent) (4.27%) and purified starch (4.27%), where the percentages are percentages by weight of the composition. The Cu-Algesic paste composition comprises 200 mg/5 g of the complex dispersed in a gel (the gel consisting of Carbopol™ (carboxyvinyl polymer) (1%), Nippasol M (n-propyl-4-hydroxybenzoate, a preservative) (0.5%), adjusted to pH ˜7.0 by addition of NaOH solution (8.5% w/v) and water, where the percentages are percentages by weight of the composition). The Cu-Algesic tablets and the Cu-Algesic paste have been administered orally to dogs and horses, respectively, without causing fatal gastrointestinal hemorrhaging.

However, it has been found that while compositions containing metal complexes of NSAIDs generally cause less adverse gastrointestinal effects than compositions containing the free NSAID, such compositions often still cause some adverse gastrointestinal effects.

In addition, it has been found that some compositions containing metal complexes of NSAIDs, including the Cu-Algesic paste, are associated with variable amounts of adverse gastrointestinal effects depending on the batch of production and the storage time prior to use.

It would be desirable to provide a composition containing a metal complex of a NSAID that causes less adverse gastrointestinal effects than prior art compositions containing the free NSAID or prior art compositions containing the metal complex of the NSAID.

The complex [Cu₂(Indo)₄(DMF)₂] included in the Cu-Algesic tablets and Cu-Algesic paste contains the ligand N,N-dimethylformamide (DMF). This ligand is toxic to humans and animals, and irritates the eyes, skin and respiratory system, causes nausea, vomiting and colic, liver damage, hepatomegaly, hypertension and dermatitis. Due to the toxicity of DMF, the regulatory authorities responsible for approving veterinary and pharmaceutical compositions for sale in Australia and other countries do not, or are reluctant to, approve compositions containing DMF for veterinary or pharmaceutical use.

### SUMMARY OF THE INVENTION

In a first aspect, the present invention provides a pharmaceutical composition comprising a metal complex of a carboxylate having anti-inflammatory activity in a pharmaceutically acceptable carrier, wherein
(1) the composition has a colloidal structure, or forms a colloidal structure when administered to a human or animal, or is immiscible with water;
(2) more than 80%, preferably more than 90%, and more preferably more than 95%, of the total amount of the carboxylate having anti-inflammatory activity in the composition is present as part of a metal complex; and
(3) less than 10% of the carboxylate having anti-inflammatory activity complexed with the metal dissociates from the metal over 12 months when the composition is stored in the absence of light at room temperature (18 to 25° C.);
   but excluding compositions comprising a metal complex containing the ligand DMF.

In a second aspect, the present invention provides a pharmaceutical composition comprising a metal complex of a carboxylate having anti-inflammatory activity in a pharmaceutically acceptable carrier, wherein
(1) the composition is immiscible with water;
(2) more than 80%, preferably more than 90%, and more preferably more than 95%, of the total amount of the carboxylate having anti-inflammatory activity in the composition is present as part of a metal complex; and
(3) less than 10% of the carboxylate having anti-inflammatory activity complexed with the metal dissociates from the metal over 12 months when the composition is stored in the absence of light at room temperature (18 to 25° C.).

Preferably less than 10% of the carboxylate having anti-inflammatory activity complexed with the metal dissociates from the metal over 18 months, more preferably less than 5% over 18 months, and most preferably less than 5% over 2 years, when the composition is stored in the absence of light at room temperature (18 to 25° C.).

The present inventors have found that the adverse gastrointestinal effects observed with the prior art compositions containing metal complexes of a carboxylate having anti-inflammatory activity, including metal complexes of a NSAID, are at least in part caused by the free carboxylate having anti-inflammatory activity released from the complex during the preparation of the composition, the storage of the composition and/or when the composition is administered to a human or animal patient. The free carboxylate released from the complex may be in the form of a carboxylate ion or an uncharged carboxylic acid depending on the pH of the surrounding medium. The present inventors have found that in the compositions of the present invention, the metal complex of a carboxylate having anti-inflammatory activity is maintained as a complex during storage and for a period of time after administration to a human or animal, and have found that the compositions of the present invention are associated with less adverse gastrointestinal effects than other compositions containing the same metal complex in which the metal complex more readily dissociates to release the carboxylate having anti-inflammatory activity.

The inventors have also surprisingly found that the compositions of the present invention when administered by routes other than oral, e.g., by topical application or by injection, are associated with less adverse gastrointestinal effects but have similar or greater anti-inflammatory efficacy, as compositions containing the free carboxylate having anti-inflammatory activity. This is surprising because the active form of a metal complex of a carboxylate having anti-inflammatory activity is the free carboxylate (as the carboxylate ion or the carboxylic acid depending on the pH of the biological fluids in which it is contained, i.e., carboxylic acid in the stomach, and carboxylate in most other fluids, tissues and organs) having anti-inflammatory activity, and it is believed that the adverse gastrointestinal affects associated with the administration of NSAIDs and other carboxylate compounds having anti-inflammatory activity by routes other than oral administration, e.g., by topical application or by injection, is caused by secondary hepatic circulation of the carboxylate having anti-inflammatory activity.

In a third aspect, the present invention provides a method for treating an inflammatory condition in a human or animal, the method comprising administering to the human or animal a therapeutically effective amount of a composition according to the first or second aspect of the present invention. The animal may, for example, be a dog, a cat, a cow, a horse, a camel, etc. The composition may be administered orally, topically, by injection, by suppository, inhalation or by some other route.

### BRIEF DESCRIPTION OF THE DRAWINGS

FIG. 1 is a graph of the gastric mucosal ulcerogenic effects in rats after oral administration of CMC (2%) solution (Control); solid-state IndoH suspended in CMC (2%) solution (10 mg kg⁻¹) (I); solid-state [Cu₂(Indo)₄(OH₂)₂] suspended in CMC (2%) solution (11 mg kg-1) (F); solid-state [Cu₂(Indo)₄(DMF)₂] suspended in CMC (2%) solution (11 mg kg⁻¹) (M); a carbopol paste containing [Cu₂(Indo)₄(OH₂)₂] (11 mg kg⁻¹) (Carb (F)); MCT paste containing [Cu₂(Indo)₄(OH₂)₂] (11 mg kg⁻¹) (MCT(F)); and MCT paste containing [Cu₂(Indo)₄(DMF)₂] (11 mg kg⁻¹) (MCT(M)). Data are presented as the means ±sem (mm²) between four rats per treatment group. A significant difference is found between the treatment group and control (P<0.05(*), P<0.01(**), and P<0.001(***)).

FIG. 2 is a graph of the mucosal ulcerogenic effects in rats after oral administration of CMC (2%) solution (Control); solid-state IndoH suspended in CMC (2%) solution (10 mg kg⁻¹) (I); solid-state [Cu₂(Indo)₄(OH₂)₂] suspended in CMC (2%) solution (11 mg kg⁻¹) (F); solid-state [Cu₂(Indo)₄(DMF)₂] suspended in CMC (2%) solution (11 mg kg⁻¹) (M); a carbopol paste containing [Cu₂(Indo)₄(OH₂)₂] (11 mg kg⁻¹) (Carb (F)); MCT paste containing [Cu₂(Indo)₄(OH₂)₂] (11 mg kg⁻¹) (MCT(F)); and MCT paste containing [Cu₂(Indo)₄(DMF)₂] (11 mg kg⁻¹) (MCT(M)); in the small intestine. Data are presented as the means ±sem (mm²) between four rats per treatment group. A significant difference is found between the control and treatment group (P<0.01(**), P<0.001(***)).

FIG. 3 is a graph of the macroscopic gastrointestinal ulcerations in rats after oral administration of MCT paste (control) (a); IndoH (10 mg/kg) (b); or an equimolar Indo and/or Cu dose of Cu-acetate (c); a physical mixture of Cu-acetate and IndoH (d); or [Cu₂(Indo)₄(DMF)₂] (e); suspended in 0.5 mL of 2% (w/v) CMC solution, or mixed in 0.5 mL of MCT paste. The results are expressed as mean ±SEM. Significant difference at P<0.01: *, from control; **, from in CMC and control; #, from IndoH.

FIG. 4 is a graph of the paw diameter change (Δmm) against time determined over 5 h following edema formation induced in rat paws by carrageenan, in rats previously orally administered a carbopol paste containing [Cu₂(Indo)₄(OH₂)₂] (11 mg kg⁻¹) and in controls. Data are presented as the means (±sem) of paw diameter change (Δmm) determined over 5 h between three rats per treatment group. A significant difference was found between the control and carbopol paste treatment animals (P<0.001 (***)).

FIG. 5 is a graph of the paw diameter change (Δmm) against time determined over 5 h following edema formation induced in rat paws by carrageenan, in rats previously orally administered an MCT paste containing [Cu₂(Indo)₄(OH₂)₂] (11 mg kg⁻¹) and in controls. Data are presented as the means (±sem) of paw diameter change (Δmm) determined over 5 h between three rats per treatment group. A significant difference was found between the control and MCT paste treatment animals at P<0.05 (*).

FIG. 6 is a graph of the paw diameter change (Δmm) against time determined over 5 h following edema formation induced in rat paws by carrageenan, in rats previously orally administered IndoH (10 mg kg⁻¹) in CMC (2%) solution and in controls. Data are presented as the means (±sem) of paw diameter change (Δmm) determined over 5 h between three rats per treatment group. A significant difference was found between the control and MCT paste treatment animals at P<0.001 (***).

FIG. 7 is a graph of the paw diameter change (A=m) against time determined over 5 h following edema formation induced in rat paws by carrageenan, in rats previously orally administered IndoH (10 mg kg⁻¹), an MCT paste containing [Cu₂(Indo)₄(OH₂)₂], or a carbopol paste containing [Cu₂(Indo)₄(OH₂)₂] (11 mg kg⁻¹)). Data are presented as the means (±sem) of % inhibition in paw diameter determined over 5 h between three rats per treatment group relative to a control group. No differences were found in the % inhibition of edema between the treatment groups at P>0.05.

### MODE(S) FOR CARRYING OUT THE INVENTION

The metal complex of a carboxylate having anti-inflammatory activity (“the metal carboxylate complex”) may be any complex comprising at least one metal ion and at least one carboxylate ligand having anti-inflammatory activity. The metal carboxylate complex may contain one or more carboxylate ligands having anti-inflammatory activity, and may contain one or more other ligands. The composition of the present invention may comprise a mixture of two or more different metal carboxylate complexes.

The carboxylate having anti-inflammatory activity may be any compound that comprises a carboxylate group and that has anti-inflammatory activity in a human or animal. Typically the carboxylate having anti-inflammatory activity is a NSAID. Typically the metal is Cu, Zn, Co or Ni, preferably Cu or Zn, and more preferably Cu.

Examples of copper complexes of NSAIDs include:

Copper-NSAID Complexes.

| Compound | Structure |
|---|---|
| [Cu₂(Sup)₄(CH₃CN)₂]^{a} | Dimer |
| [Cu₂(Sup)₄(OH₂)₂]^{a} | Dimer |
| [Cu(Tol)₂(pyridine)₂]^{b} | Monomer |
| [Cu₂(Tol)₄(dmso)₂]^{b,c} | Dimer |
| [Cu(Nap)₂(pyridine)₂]^{d} | Monomer |
| [Cu₂(Nap)₄(dmso)₂]^{c,d} | Dimer |
| [Cu(Ibu)₂(pyridine)₂]^{e} | Monomer |
| [Cu₂(Ibu)₄(dmso)₄]^{c,e} | Dimer |
| [Cu(Ibu)₂(imidazole)₂]^{e} | Monomer |
| [Cu(Ibu)₂(2-methylimidazole)₂]^{e} | Monomer |
| [Cu₂(Ibu)₄(caffeine)₂]^{e} | Dimer |
| [Cu₂(Ibu)₄(metronidazole)₂]^{e} | Dimer |
| [Cu₂(Flufen)₄L₂]^{f}, where each L is independently selected | Dimer |
| and is caffeine or papaverine. | |
| [Cu(Flufen)₂L₂]^{f}, where each L is independently selected | Monomer |
| and is nicotine, nicotinamide or N,N-diethylnicotinamide. | |
| [Cu(Nif)₂L₂]^{g}, where each L is independently selected and | Monomer |
| is 3-pyridylmethanol or water. | |
| [Cu₂(Nif)₄(dmso)₂]^{c,g} | Dimer |
| [Cu₂(Indo)₄L₂]^{h}, where each L is independently selected and | Dimer |
| is water, N,N-dimethylacetamide, N-methyl-2-pyrrolidone, | |
| tetrahydrofuran, acetonitrile, acetone or dimethylsulfoxide | |
| [Cu₂(Dic)₄L₂]ⁱ, where each L is independently selected and | Dimer |
| is water, ethanol, dimethylsulfoxide or methanol | |

where:
^{a}Suprofen = (+)-α-methyl-4-(2-thienyl-carbonyl)phenylacetic acid (SupH);
^{b}Tolmentin = 1-methyl-5-(p-toluoyl)-1H-pyrrole-2-acetic acid (TolH);
^{c}dmso = dimethylsulfoxide;
^{d}Naproxen = 6-methoxy-α-methyl-2-naphthaleneacetic acid (NapH);
^{e}Ibuprofen = (+)-α-methyl-4-(isopropylmethyl)benzeneacetic acid (IbuH);
^{f}Flufenamic Acid = (N-trifluoromethylphenyl)anthranilic acid (FlufenH);
^{g}Niflumic Acid = 2-(3-trifluoromethylphenylamino)-3-pyridinecarboxylic acid (NifH).
^{h}Indomethacin = 1-(4-chlorobenzoyl)-5-methoxy-2-methyl-1H-indole-3-acetic acid (IndoH);
ⁱDiclofenac = 2-[(2,6-dichlorophenyl)amino]phenyl acetic acid (DicH);

In this specification, the inclusion of “H” at the end of an abbreviation of the name of a compound containing a carboxylate group (e.g., any one of the carboxylate compounds listed above) is used to refer to the uncharged form of the compound, and the abbreviation without the “H” is used to refer to the deprotonated anionic form. For example, “IndoH” refers to the uncharged form of indomethacin, and “Indo” is used to refer to the deprotonated anionic form of indomethacin.

Examples of zinc complexes of NSAIDs include complexes of the general formula [Zn₂(NSAID)₄L₂] and [Zn(NSAID)₂L₂] where “NSAID” is a non-steroidal anti-inflammatory drug having a carboxylate group, and L is a monodentate ligand. Examples of such complexes include: [Zn₂(Indo)₄L₂] where L is, for example N,N-dimethylacetamide, pyridine or 1-methyl-2-pyrrolidone and [Zn(Indo)₂L₂], where L is, for example, water, alcohol as described in Syntheses and Characterization of Anti-inflammatory Dinuclear and Mononuclear Zinc Indomethacin Complexes. Crystal Structures of [Zn₂(Indomethacin)₄(L)₂] (L=N,N-dimethylacetamide, pyridine, 1-methyl-2-pyrrolidinone) and [Zn(Indomethacin)₂(L₁)₂] (L₁=Ethanol, Methanol). Zhou, Q.; Hambley, T. W.; Kennedy, B. J.; Lay, P. A.; Turner, P.; Warwick, B.; Biffin, J. R.; Regtop, H. L. Inorg. Chem. 2000, 39, 3742-3748.

Cobalt complexes of a NSAID include complexes of the general formula [Co(NSAID)₂L₂] and [Co(NSAM)₂L₄] where “NSAID” is a non-steroidal anti-inflammatory drug having a carboxylate group, and L is a monodentate ligand. Examples of such complexes include [Co(Indo)₂L₂], where L is, for example, an alcohol, and [Co(Indo)₂L₄], where L is, for example, water.

Nickel complexes of a NSAID include complexes of the general formula [Ni₂(NSAID)₄L₂] and [Ni(NSAID)₂L₄] where “NSAID” is a non-steroidal anti-inflammatory drug having a carboxylate group, and L is a mono dentate ligand. Examples of such complexes include: [Ni₂(Indo)₄L₂] where L is, for example, an alcohol; and [Ni(Indo)₂L₄], where L is, for example, water.

In some embodiments, the metal carboxylate complex is a dinuclear metal complex containing the ligand Indo. Such complexes include the complexes described in U.S. Pat. No. 5,466,824. Dinuclear metal complexes containing the ligand Indo also include complexes of the formula [Cu₂(Indo)₄L₂], where each L is independently selected and is water, N,N-dimethylacetamide, N-methyl-2-pyrrolidone, tetrahydrofuran, acetonitrile, acetone or dimethylsulfoxide. A preferred complex is [Cu₂(Indo)₄(OH₂)₂].nH₂O, when n is the number of waters of crystallisation. The number of waters of crystallisation will vary depending on the technique used to prepare the complex, and is typically from 0 to 5.

The metal carboxylate complexes referred to above may be prepared by methods known in the art. For example, Cu(II) complexes with indomethacin may be prepared as described in U.S. Pat. No. 5,466,824 or as described in Anti-inflammatory Dinuclear Copper(II) Complexes with Indomethacin. Synthesis, Magnetism and EPR Spectroscopy; Crystal Structure of the N,N-Dimethylformamide Adduct. Weder, J. E.; Hambley, T. W.; Kennedy, B. J.; Lay, P. A.; MacLachlan, D.; Bramley, R.; Delfs, C. D.; Murray, K. S.; Moubaraki, B.; Warwick, B.; Biffin, J. R.; Regtop, H. L. Inorg. Chem. 1999, 38, 1736-1744 and Preparation and Characterization of Dinuclear Copper-Indomethacin Anti-Inflammatory Drugs. Morgan, Y. R.; Turner, P.; Kennedy, B. J.; Hambley, T. W.; Lay, P. A.; Biffin, J. R.; Regtop, H. L; Warwick, B. Inorg. Chim. Acta 2001, 324, 150-161.

In some embodiments, the metal carboxylate complex is a mononuclear copper complex of the formula (1):
[Cu(η²-L¹)₂L₂]^{p} (1)
wherein “η²-L¹” is a bidentate ligand of the formula L¹: 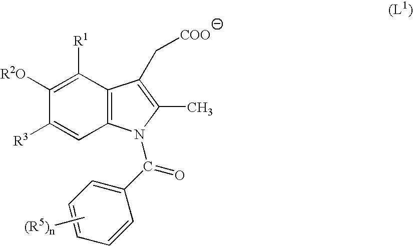
wherein:
R¹ is H or halo (i.e., Cl, F, Br or I);
R² is H; a C₁ to C₆ alkyl, an alkenyl or an alkynyl, where the C₁ to C₆ alkyl, alkenyl or alkynyl may be optionally substituted; or 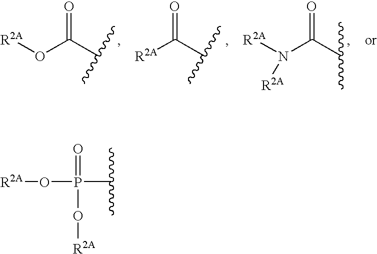
wherein each R^{2A} is independently selected from the group consisting of H, C₁ to C₆ alkyl, alkenyl, alkynyl, aryl, cycloalkyl and arylalkyl, where the C₁ to C₆ alkyl, alkenyl, alkynyl, aryl, cycloalkyl or arylalkyl may be optionally substituted;
R³ is H or halo;
each R⁵ is independently selected from the group consisting of halo, —CH₃, —CN, —OCH₃, —SCH₃ and —CH₂CH₃, where the —CH₃, —OCH₃, —SCH₃ or —CH₂CH₃ may be optionally substituted; and
n is 1, 2, 3, 4 or 5;
each L is independently selected and is a monodentate ligand,
and p is the charge of the complex.

In this specification, the term “bidentate ligand” refers to a ligand having two coordination bonds to a metal atom. Bidentate ligands include unsymmetric bidentate ligands with one longer and one shorter bond to the metal atom. In this specification, the term “monodentate ligand” refers to a ligand having a single co-ordination bond with a metal atom.

When R² is a C₁ to C₆ alkyl, an alkenyl or an alkynyl, the C₁ to C₆ alkyl, alkenyl or alkynyl may be substituted with one or more substituents. The one or more substituents may, for example, be independently selected from the group consisting of halo, —OH, —COOH and —NH₂.

When R^{2A} is a C₁ to C₆ alkyl, an alkenyl, an alkynyl, an aryl, a cycloalkyl or an arylalkyl, the C₁ to C₆ alkyl, alkenyl, alkynyl, aryl, cycloalkyl or arylalkyl may be substituted with one or more substituents. The one or more substituents may, for example, be independently selected from the group consisting of halo, —OH, —COOH and —NH₂.

When R⁵ is —CH₃, —OCH₃—SCH₃ or —CH₂CH₃, the —CH₃, —OCH₃, —SCH₃ or —CH₂CH₃ may be substituted with one or more substituents. The one or more substituents may, for example, be independently selected from the group consisting of halo, —OH, —COOH and —NH₂.

R¹ is typically H.

R³ is typically H.

R² is typically CH₃.

Each R⁵ is typically halo (i.e. F, Cl, Br or I), and n is typically 1, 2 or 3.

L¹ may, for example, be Indo.

In formula (1), L may be any monodentate ligand. L is preferably a pharmaceutically acceptable ligand. By a “pharmaceutically acceptable ligand” it is meant a ligand that does not cause any or a substantial adverse reaction when the complex is administered to a human or animal patient.

In formula (1), L may be a charged or uncharged monodentate ligand. When each L is a neutral ligand, the complex of formula (1) is neutral in charge (i.e., p is 0). However, if L is an anionic ligand, the complex of formula (1) will be charged. In some embodiments, p is 1- or 2-.

The complex of formula (1) may be present in the composition of the present invention dissolved in a component of the composition, or may be in the form of a solid, e.g., crystals of the complex. Crystals of a complex of formula (1) may include solvents of crystallisation and/or waters of crystallisation. If L is an anionic ligand, a solid of the complex of formula (1) will include cations that are counterions to the anionic complexes. Such solids, include solids having the following formulae:
Y[Cu(η²-L¹)₂L₂] (1a)
and
Y′₂[Cu(η²-L¹)₂L₂] (1b)
wherein η²-L¹ and L are as defined above for formula (1), Y is a counterion having a 2+ charge and Y′ is a counterion having a 1+ charge.

Complexes of formula (1) may for example be formed using the solvent pyrrolidine, pyrrolidine forming the ligand L in the resultant complex. Other ligands having a similar donor strength to, or a greater donor strength than, pyrrolidine can also form complexes of formula (1). In formula (1), L may, for example, be a solvent having a solvent donor number of about 30 or greater. In some embodiments, in formula (1), L is a ligand containing an N-heterocyclic group. Ligands containing an N-heterocyclic group include pyrrolidine, alkyl-substituted pyrrolidines, proline, proline derivatives, imidazole, imidazole derivatives such as substituted imidazoles or ligands containing an imidazole ring (e.g. benzimidazole), pyrrole, ligands containing pyrrole, nicotinamides and nicotinic acids. In some embodiments, in formula (1), L is an amine, e.g. NH₃ or an organic amine (e.g. diethylamine), an alcohol or an amide (e.g. diethylacetamide), or another ligand that is a strong donor such as triethylphosphate.

Complexes of formula (1) may, for example, be prepared by direct reaction of the appropriate ratios of a compound of the formula L¹H where L¹ is a group of the formula (L¹) as defined above and a copper salt such as copper(II) acetate in a solvent having a solvent donor number of about 30 or greater, the solvent forming the ligand L in the resulting complex. Complexes of formula (1) may also be prepared by adding a solvent having a solvent donor number of about 30 or greater, or adding a ligand that is not a solvent but has a similar donor strength to a solvent having a solvent donor number of about 30 or greater, to a solution of Cu(II) and L¹ in a weaker donor solvent.

Alternatively, complexes of formula (1) may be prepared by re-crystallisation of a dinuclear complex, such as [Cu₂(Indo)₄(DMF)₂], in a solvent having a solvent donor number of about 30 or greater, such as pyrrolidine, or in a solvent containing a ligand that is a strong donor.

In some embodiments, the composition of the prevent invention has a colloidal structure. In some other embodiments, the composition is formulated such that when the composition is administered to a human or animal body by the intended route of administration, a composition having a colloidal structure is formed. Such a composition typically forms a composition having a colloidal structure when the composition contacts an aqueous biological fluid in the human or animal body, for example, on contact with an aqueous fluid in the digestive tract.

A composition has a colloidal structure if it comprises a colloidal system. A colloidal system is a system in which particles of colloidal size of any nature (e.g., solid or liquid or gas) are dispersed in a continuous phase of a different composition or state.

Various colloidal systems are known and some of these are summarised below:

| Form of the | | |
|---|---|---|
| colloidal | Form of the | |
| particle | continuous phase | Type of colloidal system |
| Liquid | Gas | Liquid aerosol |
| Gas | Liquid | Foam |
| Liquid | Liquid | Emulsion |
| Solid | Liquid | Sol/suspension/hydrosol in water |
| Micelles | Liquid | Micelle solution |
| Liquid | Solid | Solid emulsion |

The composition of the present invention may comprise any of the colloidal systems referred to above. In preferred embodiments, the composition comprises micelles in an aqueous carrier or is an oil-in-water emulsion, or forms micelles or an oil-in-water emulsion when the composition is administered to a human or animal body.

Without wishing to be bound by theory, it is believed that the colloidal structure protects the metal carboxylate complex from interaction with acids or other compounds which would otherwise interact with the complex to cause the complex to dissociate, thus reducing the amount of the complex that dissociates to release the carboxylate ligand. In some embodiments of the present invention, the composition has a colloidal structure. It is believed that the colloidal structure reduces the extent to which some compounds present in the composition are able to interact with the complex, e.g. during storage of the composition, to cause the complex to dissociate. Similarly, it is believed that when such a composition is administered to a patient, the colloidal structure limits the extent to which some compounds that come into contact with the composition after it is administered are able to interact with the complex to cause the complex to dissociate before it is absorbed. For example, for compositions administered orally, it is believed the colloidal structure limits the extent to which compounds present in stomach acid are able to interact with the complex to cause the complex to dissociate before it is absorbed through the gastrointestinal tract. Similarly, for compositions administered by other routes, it is believed that the colloidal structure limits the extent to which compounds that come into contact with the composition after it is administered, e.g. strong chelators of Cu(II), such as peptides, or reductants of Cu(II), such as thiol-containing biomolecules, are able to interact with the complex to cause the complex to dissociate. In some embodiments of the present invention, the composition does not have a colloidal structure but is formulated such that when the composition is administered to a human or animal body by the intended route of administration, a colloidal structure is formed. It is believed that the colloidal structure formed when the composition is administered limits the extent to which some compounds that come into contact with the composition after administration are able to interact with the complex to cause the complex to dissociate.

In some embodiments, the composition is immiscible with water, and is thus immiscible with aqueous biological fluids. Without wishing to be bound by theory, it is believed that when such a composition is administered to a human or animal, the immiscibility of the composition with aqueous biological fluids limits the extent to which some compounds that come into contact with the composition after administration are able to interact with the complex to cause the complex to dissociate.

In some embodiments of the present invention, the composition comprises micelles in an aqueous carrier, or is in the form of an oil-in-water emulsion. When such a composition is administered to a human or animal, for example, orally, topically, by injection, to the eye, etc, the composition typically maintains the colloidal structure for some time after administration. Preferably, for a composition administered orally or topically, the colloidal structure is maintained for a sufficient time after administration of the composition for the majority, for example more than 70%, 80% or 90%, of the metal carboxylate complex to be absorbed by the body as a metal complex.

When the composition comprises micelles in an aqueous carrier, the composition typically comprises water and an amount of one or more surfactants effective to form micelles in the aqueous carrier. Any surfactants that are capable of forming micelles in the aqueous carrier, that are pharmaceutically acceptable when administered by the intended route of administration, and that do not interact with the metal carboxylate complex to cause more than 10% of the carboxylate having anti-inflammatory activity complexed with the metal to dissociate from the metal when the composition is stored in the absence of light for 12 months at room temperature (18 to 25° C.), may be used.

In one embodiment, the present invention provides a pharmaceutical composition comprising a metal complex of a carboxylate having anti-inflammatory activity, one or more pharmaceutically acceptable surfactants and water, wherein:
(1) the composition comprises micelles in an aqueous carrier;
(2) more than 80% of the total amount of the carboxylate having anti-inflammatory activity in the composition is present as part of a metal complex; and
(3) less than 10% of the carboxylate having anti-inflammatory activity complexed with the metal dissociates from the metal over 12 months when the composition is stored in the absence of light at room temperature (18 to 25° C.);
   but excluding compositions comprising a metal complex containing the ligand DMF.

An example of a composition in the form of a micelle solution is an opthalmological formulation comprising 1% w/v [Cu₂(Indo)₄(OH₂)₂] in an aqueous micelle containing polyvinyl alcohol (14 mg mL⁻¹) and povidone (6 mg mL⁻¹). In this composition, the polyvinyl alcohol acts as a solvent to dissolve the complex and as an eye lubricant, and the povidone acts as a solvent and a colloid stabiliser.

When the composition is in the form of an oil-in-water emulsion, the composition comprises one or more oils, one or more surfactants and water. Typically, the metal carboxylate complex is dissolved in the oil phase of the oil-in-water emulsion. In some embodiments, the composition comprises an oil-soluble solvent to solubilise the metal carboxylate complex in the oil phase. The oil, surfactant and water may be any combination of oil(s), surfactant(s) and water that are capable of forming an oil-in-water emulsion, that are pharmaceutically acceptable when administered by the intended route of administration, and that do not interact with the complex to cause more than 10% of the carboxylate having anti-inflammatory activity complexed with the metal to dissociate from the metal when the composition is stored in the absence of light for 12 months at room temperature (18 to 25° C.).

In one embodiment, the present invention provides a pharmaceutical composition comprising a metal complex of a carboxylate having anti-inflammatory activity, one or more pharmaceutically acceptable oils, one or more pharmaceutically acceptable surfactants and water, wherein:
(1) the composition is in the form of an oil-in-water emulsion;
(2) more than 80% of the total amount of the carboxylate having anti-inflammatory activity in the composition is present as part of a metal complex; and
(3) less than 10% of the carboxylate having anti-inflammatory activity complexed with the metal dissociates from the metal over 12 months when the composition is stored in the absence of light at room temperature (18 to 25° C.);
   but excluding compositions comprising a metal complex containing the ligand DMF.

In some embodiments, the composition further comprises a solvent to solubilise the metal carboxylate complex in the oil.

In a preferred embodiment of the present invention, the composition is a composition for oral administration comprising a metal complex of a carboxylate having anti-inflammatory activity, one or more pharmaceutically acceptable oils and one or more pharmaceutically acceptable surfactants, wherein
(1) the one or more oils and one or more surfactants are present in the composition in amounts such that following oral administration of the composition to a human or animal, the composition forms an oil-in-water emulsion on contact with aqueous fluids in the digestive system of the human or animal;
(2) more than 80%, preferably more than 90%, and more preferably more than 95%, of the total amount of the carboxylate having anti-inflammatory activity in the composition is present as part of a metal complex; and
(3) less than 10% of the carboxylate having anti-inflammatory activity complexed with the metal dissociates from the metal over 12 months when the composition is stored in the absence of light at room temperature (18 to 25° C.);
   but excluding compositions comprising a metal complex containing the ligand DMF.

A composition according to the preferred embodiment described above may optionally further comprise one or more solvents or solubilising components for increasing the solubility of the metal carboxylate complex in the composition. The solvent may, for example, be tetraglycol (IUPAC name: 2-[2-[(tetrahydro-2-furanyl)methoxy]ethoxy]ethanol; other names: 2-[2-(tetrahydrofurfuryloxy)ethoxy]ethanol; tetrahydrofurfuryldiethyleneglycol ether) or other glycofurols (also known as tetrahydrofurfurylpolyethyleneglycol ethers), polyethylene glycols, glycerol, propylene glycol, or other pharmaceutically acceptable glycols. An example of a solubilising component is a polyvinylacohol/povidone mixture. The composition may also further comprise a thickener such as Aerosil 200, clay or another inorganic filler. Such a composition may for example comprise the following ingredients in the following amounts:

| | Amount (% by weight of |
|---|---|
| Ingredient: | the composition): |
| One or more metal complexes of a carboxylate | 3 to 7 |
| having anti-inflammatory activity | |
| One or more solvents (e.g, a glycofurol) | 20 to 40 |
| One or more surfactants | 5 to 20 |
| One or more thickeners | 0 to 15 |
| Medium chain triglyceride | 40 to 60 |

A preferred composition comprises the following ingredients in the following amounts:

| | Amount (% by weight of |
|---|---|
| Ingredient: | the composition): |
| One or more metal complexes of a carboxylate | 3 to 7 |
| having anti-inflammatory activity | |
| One or more solvents (e.g, tetraglycol) | 30 ± 10% |
| One or more surfactants | 10 ± 10% |
| One or more thickeners | 5 ± 10% |
| Medium chain triglyceride | 50 ± 10% |

An example of such a composition is a composition consisting of:

| | | |
|---|---|---|
| [Cu₂(Indo)₄(OH₂)₂] complex | 5.5% | by weight |
| Tetraglycol | 30% | by weight |
| Termul 1284 | 10% | by weight |
| Aerosil 200 | 5% | by weight |
| Medium chain triglyceride | to 100% | by weight |

The present inventors have found that the oral administration of this composition causes greatly reduced adverse gastrointestinal effects compared to the oral administration of an equimolar amount of Indo in the form of Indo or a powder of the [Cu₂(Indo)₄(OH₂)₂] complex. This composition also causes less adverse gastrointestinal effects than the oral administration of an equimolar amount of Indo in the form of the prior art Cu-Algesic tablet or Cu-Algesic paste.

Suitable oils for use in compositions of the present invention include pharmaceutically acceptable vegetable or minerals oils. Suitable oils include, but are not limited to: triglycerides, particularly medium chain triglycerides, combinations of medium chain and long-chain triglycerides, combinations of triglycerides with fish oil; vegetable oils, such as, soya oil, safflower oil and sunflower oils; isopropyl myristate; and paraffins. Such oils are suitable for use in compositions for oral, injectable, or topical administration.

Suitable surfactants for use in compositions for oral or topical administration include, but are not limited to, the Sorbitan Fatty Acid Ester group of surfactants. Such surfactants comprise mono-, tri-, or partial esters of fatty acids such as oleic, lauric, palmic and stearic acids. Such surfactants include:

| | |
|---|---|
| sorbitan trioleate | (Span 85), |
| sorbitan monooleate | (Span 80), |
| sorbitan tristearate | (Span 65), |
| sorbitan monostearate | (Span 60), |
| sorbitan monopalmitate | (Span 40), and |
| sorbitan monolaurate | (Span 20). |

Other suitable surfactants include the macrogol (polyoxyethylene) esters and ethers. These surfactants include, but are not limited to, the Caster Oil Polyoxyethylene group of surfactants, such as Termul 1284. This group of surfactants comprise caster oil ethoxylate.

Other suitable surfactants in this class include the Polyoxyethylene Sorbitan Fatty Acid Esters group of surfactants, including:

polyoxyethylene (20) sorbitan monolaurate (Tween 20),

polyoxyethylene (4) sorbitan monolaurate (Tween 21), and

polyoxyethylene (20) sorbitan monooleate (Tween 80).

In some embodiments of the present invention, the composition is immiscible with water. Such compositions comprise the metal carboxylate complex in a hydrophobic pharmaceutically acceptable carrier.

Accordingly, the present invention provides a pharmaceutical composition comprising a metal complex of a carboxylate having anti-inflammatory activity in a hydrophobic pharmaceutically acceptable carrier, wherein:
(1) more than 80% of the total amount of the carboxylate having anti-inflammatory activity in the composition is present as part of a metal complex; and
(2) less than 10% of the carboxylate having anti-inflammatory activity complexed with the metal dissociates from the metal over 12 months when the composition is stored in the absence of light at room temperature (18 to 25° C.).

The hydrophobic carrier may be any hydrophobic carrier that is pharmaceutically acceptable by the intended route of administration and that does not interact with the complex to cause more than 10% of the carboxylate having anti-inflammatory activity complexed with the metal to dissociate from the metal when the composition is stored in the absence of light for 12 months at room temperature (18 to 25° C.). Suitable hydrophobic carriers for a composition for oral or topical administration include, but are not limited to, oils such as triglycerides, preferably, medium chain triglycerides, vegetable oils, such as soya oil, safflower oil and sunflower oil, isopropyl myristate and paraffins.

It is a feature of the present invention that more than 80%, preferably more than 90%, and more preferably more than 95%, of the total amount of the carboxylate having anti-inflammatory activity present in the composition is present as part of a metal complex, and that less than 10% of the carboxylate having anti-inflammatory activity complexed with the metal dissociates from the metal when the composition is stored for 12 months in the absence of light at room temperature. Compositions of the present invention having these features can be prepared by selecting suitable pharmaceutically acceptable carriers. The amount of the carboxylate present in the composition in the form of a metal complex can be readily determined by a person skilled in the art using methods known in the art, such as EPR spectroscopy.

The carrier for the composition of the present invention is selected such that the composition does not contain any ingredients or combinations of ingredients that would react with the metal carboxylate complex so as to cause more than 10% of the carboxylate having anti-inflammatory activity complexed with the metal to dissociate from the metal when the composition is stored for 12 months in the absence of light at room temperature.

Strong chelating ligands such as peptides, certain carboxylate donors, reductants such as vitamins C and E, thiolate groups such as glutathione- or cysteine-containing species, can cause metal carboxylate complexes to dissociate. Accordingly, compositions according to the present invention preferably do not comprise, or are substantially free of, peptides, carboxylate donors, reductants and thiolate groups. Preferably the composition is not strongly acidic or basic as strong acids and bases can cause metal carboxylate complexes to dissociate.

An ingredient included in some oral pharmaceutical compositions is carboxyvinyl polymer. Carboxyvinyl polymer (sold under the name Carbopol™) is included in the prior art Cu-Algesic paste. Carboxyvinyl polymer is used in pharmaceutical compositions for a variety of purposes including as a thickener. The present inventors have found that during the preparation and storage of pharmaceutical compositions containing a metal complex of a carboxylate having anti-inflammatory activity and carboxyvinyl polymer, variable amounts of the anti-inflammatory carboxylate dissociates from the complex making quality control unreliable, and accordingly, compositions according to the present invention preferably do not comprise, or are substantially free of, carboxyvinyl polymer.

Similarly, vitamin E is included in many topical opthalmological compositions and other topical compositions, as it helps repair damaged tissue in the eye or skin. However, vitamin E is a reductant, and causes metal carboxylate complexes to dissociate, and thus compositions according to the present invention preferably do not comprise, or are substantially free of, vitamin E.

When the composition is coated or is encapsulated in a capsule, the coating or capsule are selected so that the coating or capsule does not interact with the metal carboxylate complex to cause more than 10% of the carboxylate having anti-inflammatory activity complexed with the metal to dissociate when the composition is stored for 12 months in the absence of light at room temperature. For example, soft gel gelatin capsules have been shown to sequester Cu into the coating material and hence the composition of the present invention is preferably not encapsulated with a soft gel gelatin capsule.

The composition of the present invention comprises a metal carboxylate complex together with a pharmaceutically acceptable carrier. As used herein, a “pharmaceutically acceptable carrier” is a pharmaceutically acceptable solvent, suspending agent or vehicle for delivering the metal carboxylate complex to a human or animal. The carrier may be liquid or solid and is selected with the planned manner of administration in mind. The carrier must be pharmaceutically “acceptable” in the sense of being not biologically or otherwise undesirable, i.e., the carrier may be administered to a human or animal along with the active ingredient without causing any or a substantial adverse reaction.

Compositions of the present invention include those suitable for oral, rectal, nasal, topical (including buccal and sublingual), opthalmological, vaginal or parenteral (including subcutaneous, intramuscular, intravenous and intradermal) administration. The composition may conveniently be presented in unit dosage form and may be prepared by methods well known in the art of pharmacy. Such methods include the step of bringing into association the metal carboxylate complex with the carrier. The compositions of the present invention comprising an oil-in-water emulsion or micelles in an aqueous carrier may be prepared by methods known in art for preparing compositions comprising an oil-in-water emulsion or micelles in an aqueous carrier.

Typically the carrier consists of two or more ingredients. In general, the composition of the present invention is prepared by uniformly and intimately bringing into association the active ingredient with the carrier, and then if necessary shaping the product. Typically, the metal carboxylate complex and the one or more ingredients making up the carrier may be mixed in any order. However, it is preferred that the ingredients are mixed in a manner that minimises the amount of the metal carboxylate complex that dissociates during the preparation of the composition. For example, contact of the metal carboxylate complex with a strong acid or base during the preparation of the composition is preferably avoided. When the composition is prepared by adding the metal carboxylate complex to a carrier comprising micelles in an aqueous system, the mixture of the metal carboxylate complex and the carrier may be sonicated on addition of the complex to the carrier to minimise dissociation of the complex before it is incorporated into the micelles.

A composition of the present invention for oral administration may be in the form of a viscous paste, a liquid, a tablet, a capsule, a chewable composition, or any other form suitable for oral administration. If desired, the composition may be encapsulated in a suitable soft or hard capsule by techniques known in the art.

A composition for oral use may comprise one or more agents selected from the group of sweetening agents, disintegrants, lubricants, flavouring agents, colouring agents and preserving agents in order to produce pharmaceutically elegant and palatable preparations.

A chewable composition may for example comprise the metal carboxylate complex, one or more flavours, a base formulation, one or more preservatives, one or more pH modifiers, one or more desiccants and one or more fillers. The base formulation may comprise oil(s) and surfactant(s) such that when the composition is chewed and swallowed, the composition forms an emulsion in the gastrointestinal tract. The base formulation may, for example, comprise an MCT paste formulation as described in Example 3. For a chewable composition for horses, the base may comprise pre-gel starch, gelatine, flour and water. For example a chewable composition for horses may comprise the metal carboxylate complex, flavour, the base (comprising pre-gel starch, gelatine, flour and water), and other components including phosphoric acid, salt, sugar, sorbitol and/or glycerol, sorbic acid and/or potassium sorbate, benzoic acid, propionic acid and maltodextrin. A chewable composition for dogs may comprise the metal carboxylate complex, meat emulsion, an acidulant (e.g. phosphoric acid), one or more antifungal agents (e.g. benzoic acid and sorbic acid), sugar or sugar alcohol, and salt.

A composition of the present invention for topical application may comprise the metal carboxylate complex in a conventional oil-in-water emulsion, water-in-oil emulsion, or water-immiscible pharmaceutical carrier suitable for topical application. Such carriers include for example, lacrilube, cetomacrogol cream BP, wool fat ointment BP or emulsifying ointment BP. Such carriers are in the form of an emulsion or are immiscible with water.

An example of a composition for topical application is a composition comprising 0.5-2% w/w Indo as [Cu₂(Indo)₄(OH₂)₂] or [Zn(Indo)₂(OH₂)₂] in an emulsifying cream, the emulsifying cream consisting of:

| | | |
|---|---|---|
| cetomacrogol emulsifying wax | 15 | g |
| liquid paraffin | 10 | g |
| white soft paraffin | 10 | g |
| chlorocresol | 0.1 | g |
| propylene glycol | 5 | ml |
| purified and cooled water | to 100 | g. |

This composition is an oil-in-water emulsion. The cetomacrogol emulsifying wax is a surfactant (emulsifier), the combination of paraffins forms the oil phase of the emulsion, chlorocresol (4-chloro-3-methylphenol) is a preservative and propylene glycol is a solvent.

Another example of a topical composition is a composition consisting of 0.5-2% w/w [Cu₂(Indo)₄(OH₂)₂] in wool fat. This composition is immiscible with water.

Another example of a composition for topical administration is a composition comprising

| Ingredient | Amount (% by weight of the composition) |
|---|---|
| Oil Phase | |
| Iso Propyl Myristate | 9.20 |
| Arlacel 165 | 8.00 |
| GMS (NSE) | 3.00 |
| Cetyl Alcohol | 3.00 |
| Nipastat | 0.10 |
| Complex | 0.25 |
| Water Phase | |
| Water | 71.35 |
| PEG 400 | 5.00 |
| Perfume | 0.10 |
| | 100.00 |

This composition may be prepared by separately preparing the oil phase and water phase by mixing the components of each phase, and then adding the water phase to the oil phase at 65° C. The composition may alternatively be prepared by dissolution of the complex in PEG 400 with heating, adding this to the oil phase with rapid mixing until homogeneous, then adding the water and the perfume and again rapidly mixing until homogeneous.

Compositions for parenteral administration include compositions in the form of sterile emulsions and sterile aqueous or non-aqueous compositions adapted to form micelles or an emulsion when injected into a human or animal body, or which are immiscible with water (for non-intravenous injections or infusions).

An example of a composition of the present invention for subcutaneous or intramuscular injection is a composition comprising the following ingredients:

| | Amount (% by weight of |
|---|---|
| Ingredient: | the composition): |
| One or more metal complexes of a carboxylate | 1 to 7 |
| having anti-inflammatory activity | |
| One or more solvents (e.g, a glycofurol) | 10 to 50 |
| Medium chain triglyceride | to 100 |

The composition can be prepared as follows:
1. Add solvent to mixer and heat while stirring.
2. Add and dissolve metal complex. Stir until dissolved, then remove heat.
3. Add Delios V MCT oil, while stirring. Stir for 15 minutes until homogenous, then allow to cool.

An example of such a composition is a composition comprising the following ingredients:

| Ingredient: | Amount: | |
|---|---|---|
| [Cu₂(Indo)₄(OH₂)₂] complex | 40 | mg |
| Tetraglycol | 300.0 | mg |
| Delios V MCT oil | qs 1.0 | g |

Tetraglycol is the solvent; Delios V MCT oil is a medium chain triglyceride oil.

This composition is a single-phase oil and had the appearance of a dark green oil, which is immiscible in water.

An example of a composition of the present invention for intravenous injection or infusion is a composition comprising the following ingredients:

Oil Phase

| | Amount (% by weight of the |
|---|---|
| Ingredient: | oil phase): |
| One or more metal complexes of a | 1 to 7 |
| carboxylate having anti-inflammatory | |
| activity | |
| One or more solvents (e.g, a glycofurol) | 10 to 50 |
| Medium chain triglyceride | to 100 |

Aqueous Phase
Isotonic Sodium Chloride Solution

The oil phase may be prepared as follows:
1. Add solvent to mixer and heat while stirring.
2. Add and dissolve metal complex. Stir until dissolved, then remove heat.
3. Add Delios V MCT oil, while stirring. Stir for 15 minutes until homogenous, then allow to cool.

The oil phase is then mixed with the aqueous phase in a ratio of (1-3 mL oil phase): (25-50 mL aqueous phase) and the emulsion is prepared via a series of freeze-thaw degassing cycles, as described in Effect of Degassing on the Formation and Stability of Surfactant-Free Emulsions and Fine Teflon Dispersions. Pashley, R. M. J. Phys. Chem. B 2003, 107, 1714-1720.

Another example of composition for intravenous injection or infusion is a composition comprising the following ingredients in the following amounts:

Oil Phase

| | Amount (% by weight of the oil |
|---|---|
| Ingredient: | phase): |
| One or more metal complexes of a | 3 to 7 |
| carboxylate having anti-inflammatory | |
| activity | |
| One or more solvents (e.g, a glycofurol) | 20 to 40 |
| One or more surfactants | 5 to 20 |
| Oil | 40 to 60 |

Aqueous Phase
Isotonic Solution

The composition typically contains 5-30% by weight of the oil phase and 70-95% by weight of the aqueous phase. Typically the oil is a medium chain triglyceride or soya oil. The surfactant may for example be Tween 80 or soya lecithin.

In the compositions of the present invention containing a metal complex of a NSAID, typically more than 90%, preferably more than 95%, of the total amount of the NSAID in the composition is present in the composition in the form of a metal complex.

The composition of the present invention may include one or more pharmaceutically active ingredients in addition to the metal carboxylate complex.

Typically, the metal carboxylate complex constitutes about 0.1 to about 20% by weight of the composition.

The present invention also provides a method for treating an inflammatory condition in a human or animal, the method comprising administering to the human or animal a therapeutically effective amount of a composition according to the first or second aspect of the present invention. The composition may be administered orally, topically, by injection, by suppository, inhalation or by some other route.

The inflammatory condition may, for example, be rheumatoid arthritis, osteoarthritis, acute musculoskeletal disorders (such as tendonitis, sprains and strains), lower back pain (commonly referred to as lumbago), or inflammation, pain or edema following surgical or non-surgical procedures. The inflammatory condition may also be psoriasis or psoriatic arthritis.

The human or animal may be any human or animal having a disease or condition that requires treatment with a composition of the present invention. The animal is typically a mammal, and may be a non-human primate or non-primate. The mammal may for example be a companion animal such as a dog or cat, or a domestic animal such as a horse, pony, donkey, mule, llama, alpaca, pig, cow or sheep, or a zoo animal.

Suitable mammals include members of the Orders Primates, Rodentia, Lagomorpha, Cetacea, Carnivora, Perissodactyla and Artiodactyla.

For example, Artiodactyla comprises approximately 150 living species distributed through nine families: pigs (Suidae), peccaries (Tayassuidae), hippopotamuses (Hippopotamidae), camels (Camelidae), chevrotains (Tragulidae), giraffes and okapi (Giraffidae), deer (Cervidae), pronghom (Antilocapridae), and cattle, sheep, goats and antelope (Bovidae). Many of these animals are used as feed animals in various countries. More importantly, many of the economically important animals such as goats, sheep, cattle and pigs have very similar biology and share high degrees of genomic homology.

The Order Perissodactyla comprises horses and donkeys, which are both economically important and closely related.

As used herein, the term “therapeutically effective amount” means an amount effective to yield a desired therapeutic response, for example, to prevent or treat an inflammatory condition. The specific “therapeutically effective amount” will vary with such factors as the particular condition being treated, the physical condition of the human or animal, whether a human or animal is treated, the type of animal being treated, the duration of the treatment, the nature of concurrent therapy (if any), and the specific compositions employed. The dosage administered and route of administration will be at the discretion of the attending clinician or veterinarian.

In this specification, the abbreviation, “py” refers to pyridine, “Pyrro” refers to pyrrolidine, “dmso” refers to dimethylsulfoxide, and “DMF” refers to N,N-dimethylformamide.

In this specification, the term “halo” refers to fluoro, chloro, bromo or iodo.

In this specification, the term “alkyl” used either alone or in a compound word such as “arylalkyl”, refers to a straight chain, branched or mono- or poly-cyclic alkyl. Examples of straight chain and branched alkyl include methyl, ethyl, propyl, isopropyl, butyl, isobutyl, sec-butyl, tert-butyl, amyl, iso-amyl, sec-amyl, 1,2-dimethylpropyl, 1,1-dimethylpropyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, and 1,1,2-trimethylpropyl. Examples of cyclic alkyl include cyclopropyl, cyclobutyl, cyclopentyl and cyclohexyl.

In this specification, the term “cycloalkyl” refers to a saturated monocyclic or poly-cyclic alkyl having 3 to 12 carbons.

In this specification, the term “alkenyl” refers to a straight chain, branched or cyclic alkenyl. Preferably the alkenyl is a C₂ to C₂₀ alkenyl, more preferably C₂ to C₆ alkenyl. Examples of alkenyl include vinyl, allyl, 1-methylvinyl, butenyl, isobutenyl, 3-methyl-2-butenyl, 1-pentenyl, cyclopentenyl, 1-methylcyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl.

In this specification, the term “alkynyl” refers to a straight chain, branched or cyclic alkynyl, preferably a C₂ to C₂₀ alkynyl, more preferably a C₂ to C₆ alkynyl.

In this specification, the term “aryl” used either alone or in compound words such as “arylalkyl”, refers to a radical of a single, polynuclear, conjugated or fused aromatic hydrocarbon or aromatic heterocyclic ring system. Examples of aryl include phenyl, naphthyl and furyl. When the aryl comprises a heterocyclic aromatic ring system, the aromatic heterocyclic ring system may contain 1 to 4 heteroatoms independently selected from N, O and S and up to 9 carbon atoms in the ring.

In this specification the term “arylalkyl” refers to an alkyl substituted with an aryl group. An example of arylalkyl is benzyl.

The invention is described below by reference to the following non-limiting examples. It will be appreciated by persons skilled in the art that numerous variations and/or modifications may be made to the invention as shown in the following examples without departing from the spirit or scope of the invention as broadly described. The examples are, therefore, to be considered in all respects as illustrative and not restrictive.

### EXAMPLES

### Example 1

### Preparation of the [Cu₂(Indo)₄(OH₂)₂] complex

The [Cu₂(Indo)₄(OH₂)₂] complex can be prepared as described in Anti-Inflammatory Dinuclear Copper(II) Complexes with Indomethacin. Synthesis, Magnetism and EPR Spectroscopy; Crystal Structure of the N,N-Dimethylformamide Adduct. Weder, J. E.; Hambley, T. W.; Kennedy, B. J.; Lay, P. A.; MacLachlan, D.; Bramley, R.; Delfs, C. D.; Murray, K. S.; Moubaraki, B.; Warwick, B.; Biffin, J. R.; Regtop, H. L. Inorg. Chem. 1999, 38, 1736-1744, or more preferably, as described below.

Cu(II) acetate monohydrate (0.028 g, 0.140 mmol) in water (0.75 ml) was added drop wise to indomethacin (0.1 g, 0.28 mmol) dissolved in ethanol (1.75 ml) at room temperature. Warming the ethanol mildly (˜40° C.) helped solubilise the indomethacin before adding the copper acetate solution. On addition of the Cu(II) acetate monohydrate in water, a bright green complex fell out of solution immediately. This precipitate was filtered, washed with water and dried. Spectroscopic analysis shows that it was the [Cu₂(Indo)₄(OH₂)₂] complex, and EPR spectroscopy showed that it was >99% dimer.

The crystal size and colour of the [Cu₂(Indo)₄(OH₂)₂] complex was checked with a light microscope. The crystals were found to be green in colour, with a star-like shape and 50-100 microns in diameter. This size was larger (by at least an order of magnitude) than the crystals of [Cu₂(Indo)₄(OH₂)₂] prepared by the synthetic methods reported elsewhere.

### Example 2

### Preparation of bis(η²—O,O′-Indo)bis(pyrrolidine)copper(II)-2-pyrrolidine monohydrate, [Cu(Indo)₂(Pyrro)₂].2Pyrro.H₂O

[Cu₂(Indo)₄(DMF)₂] was provided by Biochemical Veterinary Research Pty Ltd. (BVR) and was purified by two recrystallisations from DMF.

Crystals that consisted of pale blue plates were grown by recrystallisation of [Cu₂(Indo)₄(DMF)₂] in pyrrolidine as the solvent. Anal. Found: C, 59.91; H, 6.32; N, 7.84; Cu, 6.01%. Calc. for CuC₅₄H₆₈Cl₂N₆O₉: C, 60.15; H, 6.36; N, 7.80; Cu, 5.84%.

Crystallographic analysis shows that the crystals are crystals of bis(η²—O,O′-Indo)bis(pyrrolidine)copper(II)-2-pyrrolidine monohydrate, [Cu(Indo)₂(Pyrro)₂].2Pyrro.H₂O. This complex may be described as a tetragonally distorted octahedron, with a four-coordinate square-planar bonding with weak off axis secondary coordination from the second ‘carbonyl’ oxygen of the carboxylate, which is bound as an unsymmetric bidentate ligand. The mononuclear Cu is bonded in a trans square-planar arrangement to two pyrrolidine nitrogen atoms at Cu—N 2.051(2) Å and via one short bond to a carboxylate oxygen atom from each of two Indo ligands at Cu—O(1) 1.9719 (14) Å. The remote carboxylate oxygen atoms bind to the Cu atoms Cu . . . O(2)=2.5696(16) Å showing weak interactions. The O(1)-Cu(1)-N(2) angle is 93.22(7)°.

### Example 3

### MCT Paste Composition

A composition of the present invention suitable for oral administration to animals or humans was prepared as described below.

The composition comprised the following ingredients:

| Ingredient: | Amount: | |
|---|---|---|
| [Cu₂(Indo)₄(OH₂)₂] complex | 55.0 | mg |
| Tetraglycol | 300.0 | mg |
| Termul 1284 | 100.0 | mg |
| Aerosil 200 | 50.0 | mg |
| Delios V MCT oil | qs 1.0 | g |

Delios V MCT oil is a medium chain triglyceride oil. Aerosil 200 is a silica based flow enhancing agent.

The composition was prepared as follows:

1 Add tetraglycol to mixer and heat to 75° C. while stirring.

2. Add and dissolve Copper Indomethacin complex. Stir until dissolved, then remove heat.

3. Add Delios V MCT oil, while stirring.

4. Add Termul 1284, while stirring.

5. Add Aerosil 200 slowly, taking care to add it to the mixing vortex while bulk is still hot. Stir for 15 minutes until homogenous, then allow to cool.

The composition was a single phase paste and had the appearance and texture of a dark green paste. When this composition is contacted with water, the composition forms an oil-in-water emulsion.

When this formulation is administered orally to a human or animal, the composition forms an oil-in-water emulsion in the digestive tract.

This composition can be administered orally to treat inflammation in animals or humans.

Similar MCT paste compositions containing other metal carboxylate complexes, such as the mononuclear complexes of formula (1) as defined above, can be prepared by the same procedure.

### Example 4

### Electron Paramagnetic Resonance (EPR) Spectroscopic Characterization of [Cu₂(Indo)₄(OH₂)₂] in Pharmaceutical Formulations

The dissociation of the [Cu₂(Indo)₄(OH₂)₂] complex results in the formation of a Cu(II) monomer that does not contain Indo and has a distinct EPR spectrum from mononuclear and dinuclear Cu(II) complexes of Indo. The synthesis of [Cu₂(Indo)₄(OH₂)₂] may result in a product containing a small amount of monomer (typically 5% or less), and this monomer may or may not contain Indo Thus a composition containing [Cu₂(Indo)₄(OH₂)₂] may or may not contain a small amount of a Cu(II) monomer containing Indo. However, as the dissociation of the dimer results in the formation of a Cu(II) monomer that does not contain Indo, the relative amount of the resultant Cu(II) monomer not containing Indo compared to the amount of the complex [Cu₂(Indo)₄(OH₂)₂] can provide an indication of the amount of dissociation of the [Cu₂(Indo)₄(OH₂)₂] complex in a pharmaceutical composition containing the [Cu₂(Indo)₄(OH₂)₂] complex, and thus the amount of free indomethacin in the composition.

The relative amounts of the Cu(II) monomer in samples of various compositions containing the complex [Cu₂(Indo)₄(OH₂)₂] were determined as described below. The compositions tested were “Cu-Algesic tablets” (containing [Cu₂(Indo)₄(OH₂)₂]), “Cu-Algesic granules” (containing [Cu₂(Indo)₄(OH₂)₂], “Cu-Algesic paste” (containing [Cu₂(Indo)₄(OH₂)₂]), and three compositions of the present invention, namely, “Cu-Algesic MCT paste” (i.e. the composition prepared as described in Example 3), “Cu-Algesic eye ointment” (1% w/w [Cu₂(Indo)₄(OH₂)₂] in lacrilube (white soft paraffin 57.3%, mineral oil (liquid paraffin) 42.5%, lanolin alcohols 0.2%) containing 1,1,1-trichloro-2-methyl-2-propanol (0.5%) as a preservative) and “Cu-Algesic eye drops” (1% w/v [Cu₂(Indo)₄(OH₂)₂] in an aqueous micelle of polyvinyl alcohol (14 mg/ml) and povidone (6 mg/ml)).

The “Cu-Algesic tablets” were the same as the prior art Cu-Algesic tablets, except that the tablets contained the [Cu₂(Indo)₄(OH₂)₂] complex rather than the [Cu₂(Indo)₄(DMF)₂] complex.

The “Cu-Algesic granules” were the same as the prior art granules used to prepare the prior art “Cu-Algesic granules” (1% w/w of CuIndo in a mixture of Aerosil (2.5% w/w) and Castor sugar (96.5%)), except that the granules contained the [Cu₂(Indo)₄(OH₂)₂] complex rather than the [Cu₂(Indo)₄(DMF)₂] complex.

The “Cu-Algesic paste” formulation used in this example contained the complex [Cu₂(Indo)₄(OH₂)₂] in a carbopol gel (the carbopol gel comprising Carbopol™ (carboxyvinyl polymer) (1%), Nippasol M (n-propyl-4-hydroxybenzoate, a preservative) (0.5%), adjusted to pH ˜7.0 by addition of NaOH solution (8.5% w/v) and water, where the percentages are percentages by weight of the composition). This composition was prepared by BVR using the same process used to prepare the prior art Cu-Algesic paste formulation containing the [Cu₂(Indo)₄(DMF)₂] complex.

The Cu-Algesic eye ointment was prepared by mixing the complex [Cu₂(Indo)₄(OH₂)₂] with the carrier.

The Cu-Algesic eye drops were prepared by mixing the complex [Cu₂(Indo)₄(OH₂)₂] with the aqueous micelle carrier and sonicating the mixture to ensure rapid dissolution of the complex into the micelle.

For each of the compositions there was no other possible source of the Cu(II) monomer other than the dissociation of the [Cu₂(Indo)₄(OH₂)₂] complex.

EPR Spectroscopy. Low temperature (4-110 K) X-band EPR spectra of the compositions containing [Cu₂(Indo)₄(OH₂)₂] were measured at X-band frequencies (˜9.5 GHz) using a Bruker EMX EPR spectrometer equipped with a standard ER4120 X band cavity, EMX 035M NMR gaussmeter, EMX 032T field controller, EMX 081 magnet power supply, Bruker EMX 048T microwave bridge control and BVT2000 variable temperature unit and Oxford Instruments E900 continuous flow cryostat (for low temperature data collection). Low temperature X-band EPR Cu(II) spectra of samples of [Cu₂(Indo)₄(OH₂)₂] containing less than 0.47 mM Cu(II) were measured using a Bruker ESP 300 spectrometer equipped with a Hewlett Packard 5352B microwave frequency counter, Bruker ER 085C magnet power supply, Bruker ER 032 magnet field control, Bruker ER 023M signal channel and Bruker ESP 1600-1048 microwave bridge controller.

All Cu(II) monomer spectra used to quantify the percent of Cu(II) monomer content relative to the total initial Cu(II) content of the samples were collected at 4 K. Either a 100-μL solution or 20 mg paste samples were placed in quartz EPR tubes (2-mm o.d., 1.5 mm i.d.) for data collection. Paste samples were placed into polyethylene tubes prior to insertion into the EPR tube. The Cu(II) monomer content relative to the total initial Cu(II) content of samples was calculated using the WINEPR data analysis program (960801; Bruker: Franzen Analytik GmbH, 1990-1996) by determining the double integral (DI) of the Cu(II) monomer spectra of the solution/paste sample and comparing this to a CuCl₂ calibration curve. Samples for the CuCl₂ calibration were prepared in Milli-Q water using grade B volumetric glassware. Glycerin (20% w/w) was added to the CuCl₂ calibration samples to produce vitrified samples suitable for EPR spectroscopy. A spectrum of the empty resonator cavity and polyethylene paste sample holder were recorded prior to any Cu(II) calibration experiment to confirm a negligible contribution of the cavity and polyethylene sample holder to the sample spectra. The absence of signal saturation for the Cu(II) monomer spectra was checked by verifying a decrease in signal intensity by the square root of the microwave power with decreasing microwave power (Weber, R. T. EMX User's Manual; Bruker Instruments, Inc.: Billerica, 1995).

Results

The distinctive resonances for Cu(II) dimers due to the spin-triplet state are characterised by the spin Hamiltonian parameters H^{⊥} (500 G), H_{z1}, (4720 G) and H_{z2} (˜5980 G). A small resonance at 3300 G due to a Cu(II) monomer fraction is also observed along with the seven-line (poorly defined) Cu-hyperfine coupling transitions on each of the g_{∥} signals (H_{z1}, and H_{z2}) (Weder, J. E.; Hambley, T. W.; Kennedy, B. J.; Lay, P. A.; MacLachlan, D.; Bramley, R.; Delfs, C. D.; Murray, K. S.; Moubaraki, B.; Warwick, B.; Biffin, J. R.; Regtop, H. L. Inorg. Chem. 1999, 38, 1736-1744). On cooling to 4 K, the Cu(II) monomer resonance increases and the Cu(II) dimer resonances (H_{⊥}, H_{z1}, and H_{z2}) disappears due to increased population of the diamagnetic ground state of the dimer, which has no EPR signal. X-band EPR spectroscopy of the samples in the 100-7000 G region were undertaken, therefore, to check for the presence of the dimer and any paramagnetic impurities.

The amount of the Cu(II) monomer in the samples, expressed as a percentage of the total amount of Cu in the composition is shown in Table 1. No quantitative results could be obtained for the Cu-Algesic tablets, or the Cu-Algesic granules in the solid state, but comparison of the EPR spectra with solutions and paste samples under the same conditions indicated that the Cu(II) content in these compositions was almost all in the form of the dimer.

The three compositions of the present invention (the Cu-Algesic MCT paste, the Cu-Algesic eye ointment and the Cu-Algesic eye drops) all contained less than 10% Cu(II) monomer fraction (Table 1). The carbopol paste formulation containing the [Cu₂(Indo)₄(OH₂)₂] complex provided by BVR Pty Ltd contained a significant fraction of Cu(II) monomer (80% of total Cu) due to the decomposition of the dimer during and subsequent to the formulation process. Other samples of the carbopol paste formulation freshly prepared by the inventors had lower amounts of the Cu(II) monomer (20-30% of total Cu) and it appears that the variability in the amounts of monomer is due to factors during the manufacture process that are difficult to control and the length time of storage after manufacture.

TABLE 1
X-band EPR spectroscopic results of the veterinary formulations of
[Cu₂(Indo)₄(OH₂)₂].

| Formulation | Cu(II) monomer (%) |
|---|---|
| Cu-Algesic tablets | —^{a} |
| Cu-Algesic granules | —^{a} |
| Cu-Algesic MCT paste | 6 |
| Cu-Algesic eye ointment | <1 |
| Cu-Algesic eye drops | 1 |
| Carbopol gel | 80 |

^{a}The technique is not applicable to dry powders, however, solid-state [Cu₂(Indo)₄L₂] (L = OH₂) contains <1% Cu(II) monomer fraction.

### Example 5

### Efficacy and Safety in Rats: a Comparison of Different Pharmaceutical Formulations

This example compared the efficacy and safety of the solid-state Test Samples and the Test Compositions described below in a series of in vivo studies for the assessment of the Test Samples and Test Compositions as anti-inflammatory agents and for their ability to induce acute gastrointestinal ulceration.

Test Samples:

Sample I=solid-state IndoH suspended in CMC (2%) solution

Sample F=solid-state [Cu₂(Indo)₄(OH₂)₂] suspended in CMC (2%) solution

Sample M=solid-state [Cu₂(Indo)₄(DMF)₂] in a micronized form suspended in CMC (2%) solution

The CMC (2%) solution is a 2% (w/v) aqueous solution of carboxymethylcellulose (CMC).

The above Samples are described below as being solid-state, meaning the complex was in the form of a solid.

Test Compositions:

A freshly prepared composition containing [Cu₂(Indo)₄(OH₂)₂] in a carbopol paste was prepared by mixing Sample F with a carbopol gel carrier (the carrier consisting of carbopol, a preservative, water and a sufficient amount of a NaOH solution to adjust the pH to ˜7.0). This composition was similar to the prior art Cu-Algesic paste, but contained the [Cu₂(Indo)₄(OH₂)₂] complex rather than the [Cu₂(Indo)₄(DMF)₂] complex.

A composition containing [Cu₂(Indo)₄(OH₂)₂] in MCT paste was prepared as described in Example 3.

A composition containing [Cu₂(Indo)₄(DMF)₂] in MCT paste was prepared in the same manner as that described in Example 3 but using [Cu₂(Indo)₄(DMF)₂] instead of [Cu₂(Indo)₄(OH₂)₂].

In this example, a reference to a micronized compound, means the compound was manufactured using the technique know as super critical fluid GAS methods that results in fine particulates of the compound (Warwick, B.; Dehghani, F.; Foster, N. R.; Biffin, J. R.; Regtop, H. L. Micronization of Copper Indomethacin Using Gas Antisolvent Processes. Ind. Eng. Chem. Res. 2002, 41, 1993-2004).

The Test Samples and Test Compositions were tested for their ability to inhibit inflammation in an inflammatory model, the carrageenan-induced paw edema model, and were also tested in a gastrointestinal ulceration model as described below.

Methods:

Samples. The composition containing [Cu₂(Indo)₄(OH₂)₂] in carbopol paste was freshly prepared for each experiment and typically exhibited only 20-30% decomposition of the dimer to Cu(II) as shown by EPR spectroscopy (Example 4).

Animals. Sprague-Dawley rats weighing 200-250 g were used throughout these studies (supplied by the laboratory animal services at the University of Sydney). Animals were housed in polypropylene cages and allowed free access to standard laboratory rat chow (Purina Rat Chow, Ralston Purina, St Louis Mo., USA) and tap water. Animals were housed in the animal care facility of the Faculty of Pharmacy at ambient temperature and humidity with a 12-h light-dark cycle. The experimental animal protocols were approved by the Animal Ethics Committee of the University of Sydney in July 1999, approval number L24/7-99/3/2972.

Gastrointestinal Ulceration: To assess gastric damage, rats (n=4 per group) were fasted overnight with free access to water prior to the oral administration (non-anaesthetized) of the Test Samples or Test Compositions via oral gavage. The controls were dosed with CMC (2%) solution. Three hours after dosing, the rats were euthanised and the stomach was excised and opened by incision along the greater curvature. The stomach was rinsed, submerged in 10% formaldehyde for 1 h and examined to determine the extent of macroscopic gastric damage. The damage is reported as the summation of the area of macroscopic ulcerations (mm²).

Rats (n=4) were allowed free access to food and water prior to and during the assessment of damage to the small intestine. The rats were orally administered (non-anaesthetized) the Test Samples or Test Compositions via oral gavage. The controls were dosed with CMC (2%) solution. At 24 h after dosing, the entire small intestine was excised and flushed with water to expel the intestinal contents. The entire small intestine was examined from 10 cm distal to the ligament of Treitz to the ileocecal junction and the damage is reported as the summation of the area of macroscopic ulcerations (mm²).

The total volume of the MCT or carbopol paste compositions administered per dose in the assessment of gastrointestinal ulceration was no more than 0.5 g.

Inhibition of Carrageenan-Induced Paw Edema: Rats were orally administered (non-anaesthetized) the Test Samples or Test Compositions via oral gavage. The control cohort was dosed solely with CMC (2%) solution. Inflammation was induced one hour after dosing with the NSAID (or vehicle), by injecting with carrageenan (0.1 mL, 2% w/v in isotonic saline) into the plantar region of the hind paw (n=3) (Winter, C. A.; Flataker, L., Pharmacol. Exp. Ther. 1965, 150, 165-171). The thickness of the paw was measured at the ventral dorsal footpad using digital calipers prior to dosing and at 3 and 5 h after carrageenan injection. The change in the measured parameter (ΔP) for thickness of paw (Δmm) at n=3- and 5-hours after carrageenan injection is given by:
ΔP=Pₜ₌ₙ−Pₜ₌₀ (I)

The percent inhibition (% inhibition) at 3- or 5-hours in the measured parameter (P) due to the treatment is given as the difference between the % increase in the value of P in the control group and the treatment group at n=3- or 5-hours, with the % increase in the value of P given by:
[(Pₜ₌ₙ−Pₙ₌₀)÷Pₙ₌₀]·100 (II)

Statistical analysis: All inhibition of carrageenan-induced paw edema and gastrointestinal ulceration data are expressed as the standard error of the mean (±sem). Comparisons among the control and treatment groups were made using one-way analysis of variance followed by a Student-Newman-Keuls t-test using the GraphPad Instat statistical program. With all analyses, an associated probability (P-value) of less than 5% (P-value<0.05) was considered significant. The calculation of the power of the experiment to compare two treatment groups with a P-value threshold of 0.05 was determined using the GraphPad StatMate program (GraphPad Instat; version 3.01 for WIN95/NT, GraphPad Software Inc., 1998).

Results

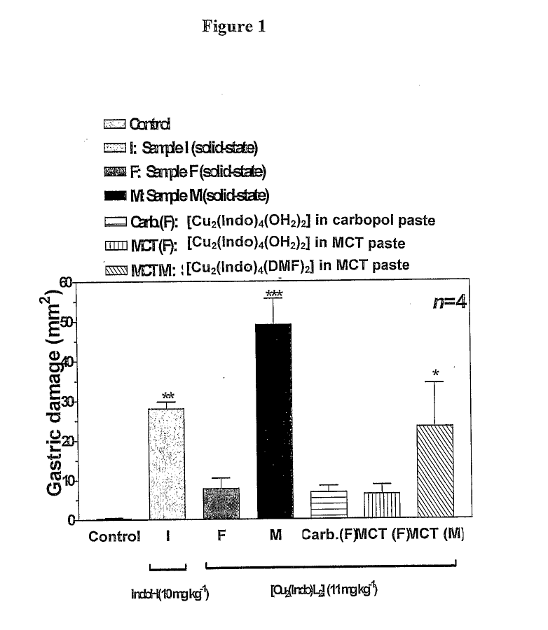

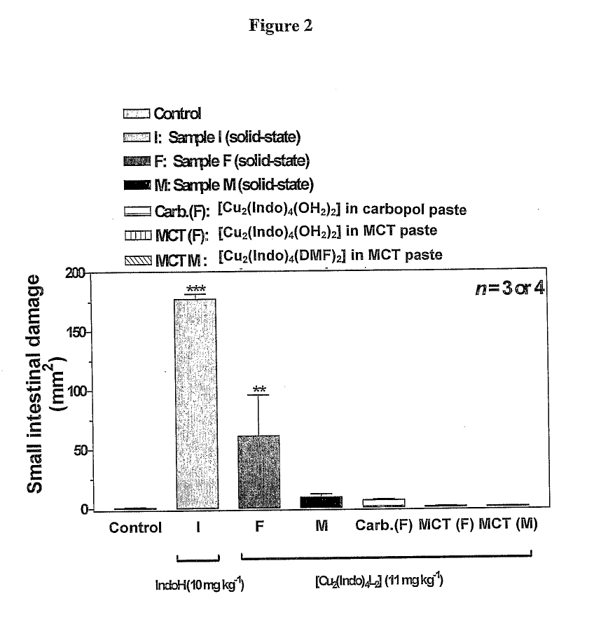

Acute GI Ulceration: Data of mean (±sem) acute gastric and small intestine ulceration (mm²) due to the various compositions are given in Table 2. Oral administration of solid-state IndoH (Sample I) (10 mg kg⁻¹) provoked significant hemorrhagic lesions in the stomach (28.0±1.7 mm², P<0.01) and small intestine (177.0±4.4 mm², P<0.001) compared to the control cohort (0.25±0.25 mm² in the stomach, and 0.5±0.5 mm² in the small intestine). While no significant ulceration (P>0.05) was found between the control and solid state [Cu₂(Indo)₄(OH₂)₂] (Sample F) treated animal in the assessment of acute gastric damage (mm²), significant ulceration was found in the assessment of acute intestinal ulceration between the control and solid-state [Cu₂(Indo)₄(OH₂)₂] (Sample F) treatment (61.0±34.5 mm², P<0.01). There was, however, a significant reduction in intestinal ulceration observed following the administration of solid-state [Cu₂(Indo)₄(OH₂)₂] (Sample F) compared to solid-state IndoH (Sample I) at P<0.001. Gastric and small intestinal mucosal ulcerogenic effects of the compositions are shown in FIGS. 1 and 2, respectively.

In contrast, solid-state micronized [Cu₂(Indo)₄(DMF)₂] (Sample M) dosed at ˜11 mg kg⁻¹ (equipotent to IndoH dosed at 10 mg kg⁻¹) produced significant gastropathy in the stomach (P<0.001, 49±7 mm²) but not in the small intestine (P>0.05, 8.7±2.9 mm²) compared to the control animals. In addition, solid-state micronized [Cu₂(Indo)₄(DMF)₂] (Sample M) caused significantly more gastropathy (49.0±6.7 mm²) than solid-state IndoH (Sample I) (28.0±1.7 mm², P<0.01) or solid state [Cu₂(Indo)₄(OH₂)₂] (Sample F) (7.8±2.7 mm², P<0.05).

The administration of the composition containing [Cu₂(Indo)₄(OH₂)₂] in MCT paste or carbopol paste resulted in significantly less gastric ulceration compared to solid-state micronized [Cu₂(Indo)₄(DMF₂)₂] (Sample M) (P<0.001). The intestinal protective effects of [Cu₂(Indo)₄(OH₂)₂] and the MCT paste of [Cu₂(Indo)₄(OH₂)₂] is highlighted by extremely significant and conclusively less intestinal ulceration (mm²) compared to solid-state IndoH at P-values<0.001. In addition, the MCT paste of [Cu₂(Indo)₄(OH₂)₂] afforded a greater protection from intestinal ulceration than the solid-state form of Sample F, with a significant reduction in intestinal ulceration (mm²) observed for the MCT paste of [Cu₂(Indo)₄(OH₂)₂] compared to the solid-state Sample F (P<0.01). Solid-state IndoH was significantly more ulcerogenic in the small intestine compared to solid-state Sample F or Sample M (P<0.001), and solid-state Sample M was significantly less ulcerogenic in the small intestine compared to solid-state Sample F (P<0.001). The fact that the MCT paste formulation reduces small intestine ulceration by an order of magnitude (back to control levels) compared to the same complex in carbopol paste is consistent with the release of more than 20% of free Indo in the carbopol paste due to degradation of the dimer. This makes the MCT pastes far superior in terms of safety in sensitive species such as dogs, particularly when compared with some of the carbopol pastes that had up to 80% of the Cu(II) dimer broken down into Cu(II) monomer and free Indo. Although these highly degraded formulations were not tested, they would have had greatly increased GI toxicity as evident by the increase in GI toxicity when much smaller amounts of free Indo were released.

Further GI Ulceration Experiments

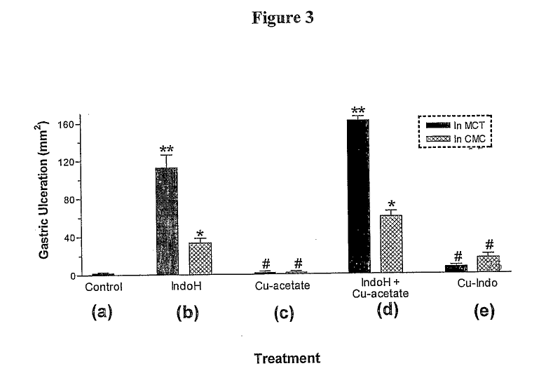

In order to illustrate the adverse effects of decomposition of the copper complex in the formulation, the gastric toxicity of IndoH, Cu-acetate, a physical mixture of Cu-acetate and IndoH and [Cu₂(Indo)₄(DMF)₂] were tested at equivalent doses of Indo (10 mg kg⁻¹) and/or Cu in the MCT paste or a 2% (w/v) aqueous solution of carboxymethylcellulose (CMC). The MCT paste formulations were prepared as described in Example 3 but using IndoH, Cu-acetate, a physical mixture of Cu-acetate and IndoH, or [Cu₂(Indo)₄(DMF)₂] instead of [Cu₂(Indo)₄(OH₂)₂]. The gastric toxicity was tested as described above. The results of these experiments are illustrated in FIG. 3. It is apparent that IndoH, or a physical mixture of Cu-acetate and IndoH in 0.5 mL MCT paste caused significantly more gastric damage than these compounds in 0.5 mL of 2% (w/v) CMC solution (P<0.01). However, there was no significant gastric ulcerations in rats treated with [Cu₂(Indo)₄(DMF)₂] in MCT paste, or Cu-acetate in 0.5 mL of 2% (w/v) CMC solution or mixed in 0.5 mL of MCT paste compared to the control. Moreover, while IndoH or a physical mixture of Cu-acetate and IndoH in 0.5 mL MCT paste were more GI toxic than the same test samples in CMC, the opposite was the case for [Cu₂(Indo)₄(DMF)₂] where there was less GI toxicity for the MCT formulation compared to the CMC formulation.

In further experiments designed to test the toxicity of solutions of the Cu complex, the [Cu₂(Indo)₄(DMF)₂] complex was dissolved in DMF and gastric toxicities were assessed. For the control (DMF only), 1 mm² of gastric ulceration was observed in each of the two rats tested, whereas [Cu₂(Indo)₄(DMF)₂] (10 mg kg⁻¹ dose of Indo) in either DMF alone, or DMF containing 2% CMC, resulted in large amounts of ulceration (>200 mm² and 100 mm², respectively). Similar results would be expected using other complexes, including [Cu₂(Indo)₄(OH₂)₂], in a non-colloidal solution. The results of this experiment demonstrate that the administration of a metal carboxylate complex in solution, rather than in a composition of the present invention, makes the complexes highly GI toxic, due to their rapid decomposition on contact with gastric juices.

The further experiments using DMF as the carrier and comparisons of different forms of Cu and Indo in CMC and MCT formulations described above were performed under animal protocols that were approved by the Animal Ethics Committee of the University of Sydney in Jun. 3, 2002 and Dec. 5, 2003, approval numbers, L07/6-2002/2/3575 and L07-1/2004/3/3846, respectively

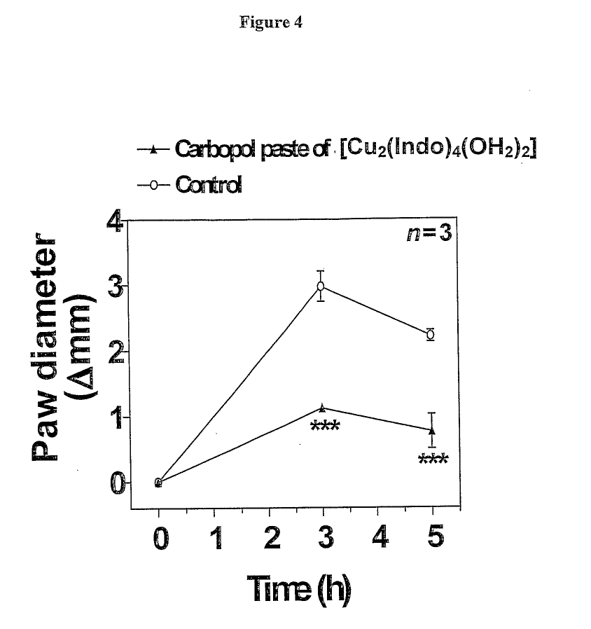

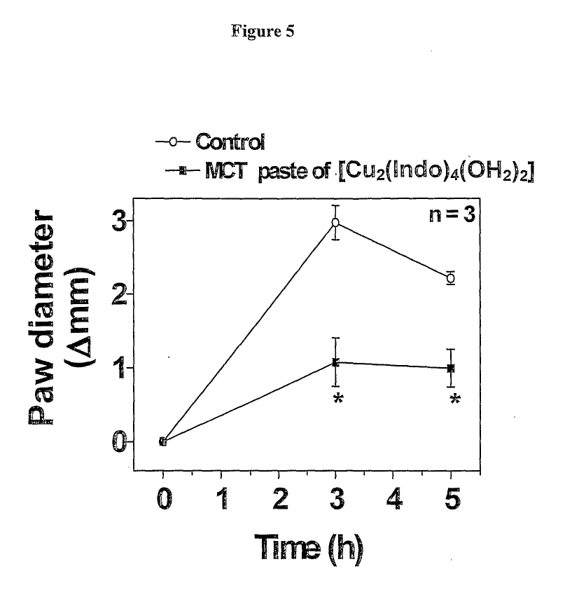

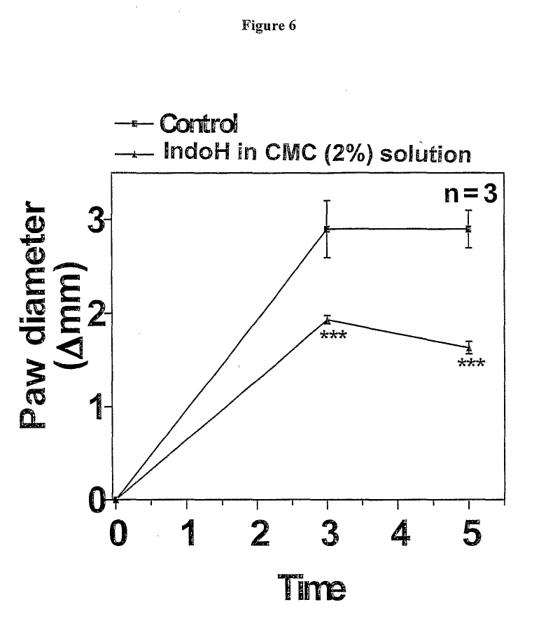

Inhibition of Carrageenan-Induced Paw Edema: The intraplantar injection of carrageenan (0.1 mL of 2% solution) elicited acute hind-paw inflammation and caused a time-dependent increase in paw edema as measured by rat paw diameter change (Δmm). A peak inflammatory response was observed at 3 h after the injection (FIGS. 4, 5 and 6). No significant difference (P>0.05) was observed in paw edema change (Δmm) between the control groups at 3- and 5-hours post-carrageenan injection.

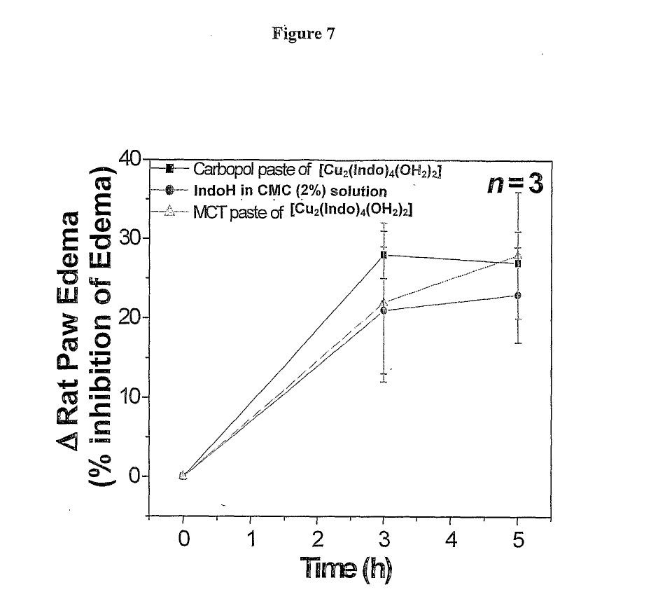

Treatment of animals with IndoH (10 mg kg⁻¹) in CMC (2%) solution suppressed the paw diameter change (Δmm); with the % inhibition in paw diameter change relative to the control cohort being 21(8) % and 25(6) % at 3- and 5-hours, respectively (Table 3 and FIG. 7). Likewise, the carbopol and MCT pastes of [Cu₂(Indo)₄(OH₂)₂] (11 mg kg⁻¹) prepared from Sample F resulted in percent inhibition in paw diameter change relative to the control cohort at 3- and 5-hours of 28(3) % (3-hr) and 27(4) % (5-hr) for the former and 22(10) % (3-hr) and 28(8) % (5-hr) for the latter (Table 2 and FIG. 7). The greater the value of the % inhibition in rat paw diameter change, the greater is the anti-inflammatory effect of the treatment. There was a significant difference in paw diameter change (Δmm) as a result of treatment with MCT (P<0.05) or carbopol (P<0.001) pastes of [Cu₂(Indo)₄(OH₂)₂] compared to control at 3- and 5-hours post-carrageenan injection (FIG. 7). This result indicates both the carbopol and MCT pastes elicited anti-inflammatory effects, despite the different amounts of Cu(II) dimer relative to free Indo in the two compositions.

A significant difference was found between the control cohort and the IndoH (10 mg kg⁻¹) in CMC (2%) solution treated group. No significant difference was found, however, between the anti-inflammatory efficacy of the MCT and carbopol paste treatments and IndoH in CMC (2%) solution treatment as assessed by changes in rat paw diameter (% inhibition) at 3 and 5-hours post-carrageenan injection (P<0.05). A plot of the anti-inflammatory efficacy of the treatments (as represented by % inhibition of edema) is shown in FIG. 7.

### DISCUSSION AND CONCLUSION

The present study showed that supratherapeutic doses of a non-micronized solid-state [Cu₂(Indo)₄(OH₂)₂] (Sample F) was significantly less toxic in both the stomach and intestine than equimolar doses of the parent NSAID (IndoH); with the complex affording a significant anti-inflammatory effect similar to IndoH. Furthermore, the nature of the pharmaceutical formulation influenced the extent of the complex's GI-protective effect, with the incorporation of the complex into a GI protective composition of the present invention further augmenting a significant reduction in GI toxicity compared to IndoH. It is known that formulation is important when considering not only the efficacy and toxicity of the drug, but also its pharmacokinetics. Since the ultimate utility of most NSAIDs for treatment of inflammation is limited by its side effects, the minimisation of such side effects as described herein is a critical step in the clinical application of NSAIDs for the treatment of inflammation.

Whilst particle shape can influence surface area, the most probable cause of the enhanced GI toxicity in the stomach of the rats following administration of the solid-state micronised dose of [Cu₂(Indo)₄(DMF)₂] (Sample M) compared to solid-state factory grade [Cu₂(Indo)₄(OH₂)₂] (Sample F) is increased degradation of Sample M to free IndoH due to an increased surface area of the administered dose. The increase in the surface area of the administered dose of Sample M is a result of the smaller mean surface area of the micronized particles manufactured by the super critical fluid GAS system compared to the factory grade aggregates (Sample F) produced from the conventional factory grade process. There was a significant difference in the values of both the surface area (μm²) and circularity parameter (P<0.001) of the particle sizes of Sample M compared to Sample F.

The acute stomach toxicity of solid-state micronized [Cu₂(Indo)₄(DMF)₂] (Sample M) was significantly reduced by the formulation of the particles into an MCT paste composition, probably due to a protective gastric barrier afforded by the paste and the stability of the complex within the formulation.

Whilst the Cu(II) dimer complex of Indo is retained in the MCT paste compared to the carbopol paste, no significant difference was found between the anti-inflammatory efficacy of the MCT or carbopol paste treatments as assessed by changes in rat paw diameter (Δmm) in both treatment groups at 3 and 5-hours post-carrageenan-induced paw edema (P>0.05). This may be due to the lack of sufficient sensitivity of the anti-inflammatory assay to differentiate between the efficacies of the treatments, achievement of equivalent plasma and tissue concentrations between the formulations or the administration of a supratherapeutic dose being near the effective maximum anti-inflammatory dose. Nonetheless, both the MCT (2% Cu(II) monomer content) and carbopol (20% to 30% Cu(II) monomer content) pastes of Sample F afforded equipotent anti-inflammatory activity compared to the control group.

The acute direct toxicity of NSAIDs in the small intestine is highlighted by the highly significant intestinal ulceration observed following the administration of solid-state IndoH (Sample I) compared to the control cohort. The present study confirmed a highly significant ulcerogenic-sparing activity in the intestine of solid-state [Cu₂(Indo)₄(OH₂)₂] compared to solid-state IndoH (P value<0.001). This result is contrary to an early report by others of unaltered ulcerative damage by IndoH when given as a Cu(II) complex (Boyle, E.; Freeman, P. C.; Goudie, A. C.; Mangan, F. R.; Thomson, M., J. Pharm. Pharmac. 1976, 28, 865-868). Furthermore, there was an additional increase in small intestine protection when the [Cu₂(Indo)₄(OH₂)₂] complex was formulated into an MCT paste rather than administered to the animals as a powdered dose form of [Cu₂(Indo)₄(OH₂)₂]. Instability of [Cu₂(Indo)₄(OH)₂] in the stomach is best avoided in order to prevent degradation of the ulcerogenic-sparing Cu complex of Indo to free IndoH (which has significant ulcerogenic side-effects in both the stomach and intestine). This was evidenced by the enhanced gastropathy caused by the micronized solid-state [Cu₂(Indo)₄(DMF)₂] compared to factory grade solid-state [Cu₂(Indo)₄(OH₂)₂], which was ameliorated by its formulation into an MCT paste. The need to protect the complex from acid-catalyzed decomposition in the stomach is also highlighted by the very high GI toxicity of the complex dissolved in DMF, where the complex will react immediately and completely on oral administration when it reaches the gastric juices.

The administration of the solid-state micronized [Cu₂(Indo)₄(DMF)₂] complex of Indo caused significantly less toxicity in the small intestine than solid-state IndoH or solid-state [Cu₂(Indo)₄(OH₂)₂], possibly due to the enhanced absorption of free IndoH from the stomach following the disintegration of the Cu(II) dimer complex. Enhanced bioavailability of drugs due to GI mucosal damage is reported elsewhere, e.g., following administration of “permeability enhancers” such as 5-methoxysalicylate (Peters, G. E.; Hutchinson, I. E. F.; Hyde, R.; McMartin, C.; Metcalfe, S. B. J. Pharm. Sci. 1987, 76, 857). The acute gastric toxicity of the micronized solid-state [Cu₂(Indo)₄(DMF)₂] compared to solid-state IndoH was no doubt due to the increased surface area of the micronized dose of [Cu₂(Indo)₄(DMF)₂] (particle size 2.98±1.24 μm²) compared to IndoH (particle size 226±19 μm²).

The acute gastropathy associated with the administration of micronized [Cu₂(Indo)₄(DMF)₂] further highlights the importance of characterizing the pharmaceutical nature of the veterinary formulation and ensuring the retention of the Cu(II) complex in the GI tract. Whilst the current work confirmed the GI-sparing toxicity of the Cu(II) complex of Indo compared to the parent NSAID as reported by others (Sorenson, J. R. J., Prog. Med. Chem. 1989, 26, 437-568), the carrageenan-induced rat paw edema results are unable to verify an enhanced anti-inflammatory efficacy of the Cu-Indo complex compared to IndoH.

In summary, the nature of the composition containing a metal complex of Indo does not appear to have a large effect on the anti-inflammatory efficacy of the complex at saturation, but it has a dramatic effect on the GI toxicity. The micronized [Cu₂(Indo)₄(DMF)₂] complex is highly GI toxic, like IndoH, since its large surface area facilitates acid-induced breakdown to free IndoH in the stomach. Larger crystals induce considerably less ulceration, but the greatest GI protection is obtained with the composition of the present invention ([Cu₂(Indo)₄(OH₂)₂] in the MCT paste). While there is no difference in gastric protection between fresh carbopol pastes containing only 20% to 30% dissociation of the complex and MCT pastes, there is an order of magnitude decrease in small intestine toxicity in the MCT paste over the carbopol pastes, no doubt due to the increased levels of free indomethacin in the carbopol paste. Moreover, the Cu-Indo dimer degradation during formulation and soon afterwards in the carbopol pastes shows a large variation between batches, as observed by visual changes in the colour of the paste and by EPR spectroscopy determination of the dimer content. This degradation leads to an increase in free Indo in the composition, which makes the carbopol pastes less suitable as a pharmaceutical formulation, especially for the treatment of species that are sensitive to free IndoH, such as dogs. Even for the carbopol formulations with the least decomposition of dimer (20% to 30%), the carbopol paste is more GI toxic than the MCT paste composition. The carbopol batches with up to 80% decomposition of the Cu(II) dimer, with consequential release of free Indo, are expected to be almost as GI toxic as similar formulations with Indo only.

The importance of maintaining the integrity of the complex is illustrated in the comparisons of the data on the GI toxicities of IndoH, Cu-acetate, a physical mixture of Cu-acetate and IndoH and [Cu₂(Indo)₄(DMF)₂] at equivalent doses of Indo (10 mg kg⁻¹) and/or Cu in 2% CMC or the MCT paste. These results show clearly that a physical mixture of a copper salt and IndoH in the appropriate formulations cannot impart the gastroprotective effects of the Indo complexes. In fact, the physical mixtures were more GI toxic than IndoH alone, which highlights the requirements to not only have formulations where the integrity of the Cu complex is maintained, but the formulation has to be sufficiently stable on oral ingestion to prevent the acid-catalyzed decomposition of the drug before it is absorbed. Thus the compositions of the present invention are superior to the carbopol paste formulation, since there was considerable variability in the amount of decomposition of the metal complex in factory manufactured batches of the carbopol paste formulation (20-80%), which led to adverse reactions in animals treated with the more degraded samples.

These results also illustrate other facets of the MCT paste composition and similar compositions of the present invention that make them superior to delivery of the drugs formulated in CMC for instance. The complexes suspended in CMC are less gastroprotective than in the MCT paste formulation, because the CMC is less able to protect the drug from acid decomposition in the stomach. This is illustrated by the fact the [Cu₂(Indo)₄(OH₂)₂] complex in the MCT paste formulation at a high therapeutic dose has only approximately 8% of the gastric toxicity of an equivalent dose of IndoH, whereas the toxicity of a suspension in CMC is reduced by only 50% when Indo is delivered in the form of the complex as opposed to free IndoH. This illustrates that almost all of the ([Cu₂(Indo)₄(OH₂)₂] complex delivered in the MCT paste formulation is absorbed intact, whereas approximately half of the complex suspended in CMC decomposes before it is absorbed.

The reason, underlying the smaller absolute differences in toxicity when the complex is delivered in CMC and the MCT paste formulation is that the MCT paste formulation is superior to CMC in delivering the drugs. This is illustrated by the potency of the drugs in the two formulations, thus while IndoH has a similar efficacy (maximum anti-inflammatory effect) when it is delivered in the MCT paste formulation and the CMC formulation, the potency (i.e., as measured by the concentration that produces 50% of the maximum efficacy, ED₅₀ value) is superior in MCT paste (ED₅₀ of 1 mg kg⁻¹) versus CMC (ED₅₀ of 2.8±1.7 mg kg⁻¹). Thus IndoH and ([Cu₂(indo)₄(OH₂)₂] in the MCT paste formulation are absorbed much more efficiently than in the CMC formulation, which leads to the much higher gastric toxicity of IndoH in the MCT paste formulation compared to the CMC formulation. Thus stabilisation of the drug in the stomach as a colloidal emulsion, as in the case of the MCT paste formulation, leads to the dual desirable characteristics of increased potency (lower therapeutic dose) and decreased toxicity. The greatly enhanced therapeutic window that results from such formulations, eliminates most of the undesirable side-effects that have limited the use of potent NSAIDs such as IndoH.

Without wishing to be bound by theory, the present inventors believed that the marked reduction in GI toxicity for the oral administration compositions of the present invention containing copper indomethacin complexes compared to the oral administration of Indo, as evidenced by the results in this example, is due to three separate and additive contributions, which impart the high safety as described below.
(i) The compositions of the present invention result in at least some of the copper indomethacin complex being absorbed in the gastrointestinal tract before the complex dissociates, and thus the compositions of the present invention minimise the amount of free Indo/IndoH that is available to interact with the COX-1 enzymes in the mucosa in order to cause primary GI toxicity.
(ii) Metal complexes of Indo are more lipophilic than Indo, and therefore copper indomethacin complexes are absorbed more readily than IndoH, and this results in less time for interaction with COX-1 enzymes in the mucosa.

(iii) The angiogenic nature of the Cu in copper complexes of Indo promotes wound healing and hence provides an anti-ulcerogenic effect, whereas IndoH alone actually retards angiogenesis and, hence, promotes ulceration, hence, IndoH promotes ulceration from secondary circulation of Indo, while Cu helps repair and prevent ulceration from secondary circulation of Indo.

TABLE 2
Data of mean acute gastric and small intestine ulceration ± sem (mm²).
Gastric ulceration (mm²)

| | | [Cu₂(Indo)₄(OH₂)₂] | [Cu₂(Indo)₄(DMF)₂] | Carbopol paste of | MCT paste of | MCT paste of |
|---|---|---|---|---|---|---|
| Control | IndoH solid-state | solid-state | solid-state | [Cu₂(Indo)₄(OH₂)₂] | [Cu₂(Indo)₄(OH₂)₂] | [Cu₂(Indo)₄(DMF)₂] |
| (n = 4) | (Sample I) (n = 4) | (Sample F) (n = 4) | (Sample M) (n = 3) | (n = 4) | (n = 4) | (n = 4) |
| 0.25 ± | 28.0 ± 1.7 | 7.8 ± 2.7 | 49.0 ± 6.7 | 6.8 ± 1.7 | 6.3 ± 2.4 | 23.3 ± 10.9 |
| 0.25 | | | | | | |

Small intestine ulceration (mm²)

| | | [Cu₂(Indo)₄(OH₂)₂] | [Cu₂(Indo)₄(DMF)₂] | Carbopol paste of | MCT paste of | MCT paste of |
|---|---|---|---|---|---|---|
| Control | IndoH solid-state | solid-state | solid-state | [Cu₂(Indo)₄(OH₂)₂] | [Cu₂(Indo)₄(OH₂)₂] | [Cu₂(Indo)₄(DMF)₂] |
| (n = 4) | (Sample I) (n = 3) | (Sample F) (n = 3) | (Sample M) (n = 3) | (n = 4) | (n = 4) | (n = 4) |
| 0.50 ± | 177.0 ± 4.4 | 61.0 ± 34.5 | 8.7 ± 2.9 | 6.0 ± 0.9 | 0.5 ± 0.3 | 0.25 ± 0.25 |
| 0.50 | | | | | | |

TABLE 3
The percent inhibition in rat hind-paw diameter change due to treatment
3- and 5-hours post intraplantar injection of carrageenan
(0.1 mL of 2% solution).

| Treatment | 3-hr | 5-hr |
|---|---|---|
| Indomethacin (10 mg kg⁻¹) in CMC (2%) solution | 21(8)% | 25(6)% |
| [Cu₂(Indo)₄(OH₂)₂] (11 mg kg⁻¹) in CMC (2%) | 30(4)% | 31(3)% |
| solution^{a} | | |
| MCT paste of [Cu₂(Indo)₄(OH₂)₂] (11 mg kg⁻¹)^{a} | 28(3)% | 27(4)% |
| Carbopol paste of [Cu₂(Indo)₄(OH₂)₂] (11 mg kg⁻¹)^{a} | 22(10)% | 28(8)% |

^{a}Sample F (factory grade).

### Example 6

### Cu-Algesic Forte Paste: Bioequivalence Study

This example compared the bioavailabilty of indomethacin in a composition of the present invention (Test Composition B) with a composition similar to the prior art Cu-Algesic paste formulation containing the same metal complex of indomethacin (Test Composition A). Both Test Composition A and Test Composition B contained the complex [Cu₂(Indo)₄(OH₂)₂]. Test Composition B was the composition prepared as described in Example 3 (the MCT paste formulation). Test Composition A was a composition comprising the complex in a carbopol paste. Test Composition A is similar to the prior art Cu-Algesic paste formulation, but contains the complex [Cu₂(Indo)₄(OH₂)₂]. The prior art Cu-Algesic paste formulation contains the complex [Cu₂(Indo)₄(DMF)₂] in a carbopol paste, and is a commercially available paste formulation used in Australia for the treatment of animals. The prior art Cu-Algesic paste formulation, whilst efficacious, is variable in its efficacy and tolerability.

1.1 Sample Analysis and Statistical Analysis

Centre for Heavy Metals Research, School of Chemistry, University of Sydney.

1.2 Study Location/Test Facility

Rural Veterinary Centre, University of Sydney Large Animal Hospital, Werombi Rd., Werombi.

1.3 Study Schedule

Experimental Start Date:

27/11/01 (1^{st} treatment)
17/12/01 (2^{nd} treatment)
   Experimental End Date:
25/12/01 (final sampling time)
15/5/02 (final sample analysis)
   2. Materials and Methods
   2.1 Study Design

The study design was based on the FDA Guidance for Industry Bioequivalence Guidance.

2.1.1 Treatment Groups

Two groups of 4 horses.

2.1.2 Experimental Design/Blocking:

The two pastes, Test Composition A and Test Composition B were administered in the experiment using a randomised cross-over design as outlined in the following table:

| Horse | Treatment period 1 | Treatment period 2 |
|---|---|---|
| 1 | A | B |
| 2 | A | B |
| 3 | A | B |
| 4 | A | B |
| 5 | B | A |
| 6 | B | A |
| 7 | B | A |
| 8 | B | A |

2.1.3 Wash-Out Period

A wash-out period of 20 days was used, based on the FDA Guidance which recommends a wash-out period of 10× the plasma half-life to provide for 99.9% of the administered dose to be eliminated from the body.

2.1.4 Randomisation and Allocation Procedures

Horses were randomly assigned to each group.

2.1.5 Blinding

As the experiment involved only blood collection, only the analyst was blinded, serum sample tubes were labelled with horse number only.

2.2 Animal Selection and Identification

2.2.1 Details of Animals

Eight standard-bred mares aged between 5 and 8 years were used in the experiment.

2.2.2 Preparation of Animals

All horses were given anthelmintic treatment and tetanus prophylaxis 7 days prior to the trial.

2.3 Animal Housing and Management

2.3.1 Housing and Management

Horses were placed in pairs in 4 dirt yards with secure pipe fencing. Horses were returned to the paddock for a washout period of 20 days. The horses were brought up into the yards the night before the second treatment period.

2.3.2 Feed

All horses had free access to food (hay) and water throughout the experimental period. Horses were fed on the night prior to trial and were fed immediately after drug administration at each of the two drug administration days.

2.3.3 Animal Handling

Veterinarians or staff at Rural Vet Centre.

2.3.4 Removal of Subject(s) from the Study

Horses that have developed any illness or trauma requiring medication were to be removed from the study. No horses had to be removed from the study.

2.3.5 Concurrent Therapies

No other medication, particularly NSAIDs, was permitted.

2.3.6 Owner Consent

The horses were owned by the Rural Vet Centre.

2.4 Treatments

Test Composition A

| Active Ingredient: | | |
|---|---|---|
| Formulation: | Copper Indomethacin | |
| Copper Indomethacin | 40.0 | g/kg |
| Carbopol | 10.0 | g/kg |
| Methyl Hydroxybenzoate | 3.0 | g/kg |
| Propyl Hydroxybenzoate | 1.0 | g/kg |
| Potable Water | Qs ad 1 | kg |

Test Composition B

| Active Ingredient: | | |
|---|---|---|
| Formulation: | Copper Indomethacin | |
| Copper Indomethacin | 55.0 | g/kg |
| Tetra Glycol | 300.0 | g/kg |
| Termul 1284 | 100.0 | g/kg |
| Aerosil | 50.0 | g/kg |
| Delios (MCT) | Qs ad 1 | kg |

2.4.3 Drug Administration
Dosing regimen: 0.8 mg/kg, calculate dose based on weight
Route of administration: Oral
Wash-out period: 20 days
   2.5 Test Samples
   2.5.1 Blood Sample Collection

On the morning of the trial, a 14 gauge over the needle catheter and T port was placed aseptically into the left jugular vein and secured with quick set glue and suture. The catheters were flushed with heparinized saline.

A 20-ml blood sample (time 0) was collected immediately prior to administration of the paste and placed into 2 serum tubes. Further samples were taken at 1 hr intervals for 18 hours and then at 2 hour intervals until 24 hours (0, 1, 2, 3 . . . 18, 20, 22, 24). Catheters were flushed after each collection period with heparinized saline. Catheters were removed after the 24 hr collection and further samples were taken aseptically by venipuncture at 48, 96 and 192 hours post administration of each test composition. A 20-ml aliquot (one blood tube full) was collected at each sampling time into serum tubes labelled with horse ID number/name, sampling time (0, 1 hr etc.) and date. Sample collection time was recorded on a data sheet and each time point was initialized.

Horses were returned to the paddock for a washout period of 20 days. The horses were brought up into the yards the night before the second treatment period. Catheters were placed in the right jugular vein the morning of the trial. Horses that received Test Composition A in the first treatment period received Test Composition B in the second treatment period and horses receiving Test Composition B in the first treatment period received Test Composition A as outlined in the table. Blood samples were collected at the same time intervals and the serum aseptically harvested and frozen for assay.

2.5.2 Sample Handling

All serum samples were immediately spun down in a centrifuge at 3000 rpm for 10 minutes and the serum aseptically collected into labelled specimen tubes and frozen until assayed.

Samples were stored in a secure location in the freezer until transported to University of Sydney, School of Chemistry for analysis.

2.6 Sample Analysis, HPLC

2.6.1 Materials and Reagents

Indomethacin for standards, Mefanamic acid and Acemethacin for internal standards were of pharmaceutical grade (Sigma Pharmaceuticals). Methanol and acetonitrile were of HPLC grade (Aldrich). Acetic acid was analytical grade (Aldrich). Purified water was obtained using a Milli-Q reagent water system (Millipore).

2.6.2 Gradient HPLC Analysis

The analysis was performed on a Hewlett-Packard HPLC Series HP1100 with Diode-Array UV/VIS detector. Separation was achieved using a 5-μm RP-ZORBAX XDB-C₁₈, (250×4.6 mm I.D) column (Hewlett-Packard) equipped with a 5-μm ODS guard column (Hewlett-Packard). The flow rate was 1 ml/min and the monitoring wavelength was 254 nm and 270 nm. A linear gradient, from 60% to 85% solvent B over 22 min was performed (solvent A: 0.5% acetic acid in water; solvent B: 0.5% acetic acid in a mixture of acetonitrile and methanol 1:1).

2.6.3 Sample Preparation

Horse plasma samples (1 ml) buffered to pH 3.5, were deproteinized with 5 ml acetonitrile, centrifuged at 3000 g. The supernatant were evaporated to dryness under nitrogen flow and reconstituted in mobile phase (100 μl) and aliquots of 20 μl were injected.

2.6.4 Calibration Curves

The calibration curve for indomethacin in plasma was constructed by spiking blank horse plasma with known concentrations of 5, 10, 20, 50, 100, 200, 500, 1000, 1500 and 2000 ng/ml. A typical calibration curve of indomethacin was described by the equation y=6.88636e-1x+2.09520e-1 (r=0.99918). In the equation, y represents the peak-area ration of the analyte to I.S., where x correspond to plasma concentration in ng/ml.

2.6.5 Quality Control

Quality control was performed at level 10, 100, 1000 ng/ml during HPLC analysis of each horse plasma sample.

b 2.7 Statistical Analysis

The data were analysed to determine AUC values with WinNonlin version 1 software, using a non-compartment model. The area under plasma concentration vs time was estimated by linear/log trapezoidal approximation from 0 to 24 h, 0 to 48 h, 0 to 96 or 0 to 192 h.

In all horses the level of indomethacin had reached baseline levels after 24 hours and, therefore, all discussion hereafter is based on the analysis of the data up to the 24-hour time-point. Statistical analysis of the data was carried out using Student's t tests according to the FDA guidelines and using non-parametric tests which are more appropriate for a data set of this type. Analysis involved comparing the AUC for each of the two test compositions (A & B), bioequivalence requires a non-significant difference between AUC for each formulation.

3. Results and Discussion

The data for all horses showed a pattern with multiple peaks in the indomethacin levels with the peak level occurring at 1-9 hours and the baseline level reached after 24 hours. Substantial inter-horse variation is observed, particularly in the AUC values suggesting variable uptake of the drug from the stomach. It is notable that the pattern of inter-horse variation was similar for the two drugs suggesting that the variability is due to the animal and not external variables.

Using a Student's t test, analysing the average values for each formulation, the differences between the AUC values for Test Compositions A & B are found to be not significant at the 95% confidence level (or at the 90% confidence level). In accord with this there is no consistent trend in these values for individual horses.

Using a non-parametric test, which concentrates on the differences for individual horses, the differences are also not significant. Emphasising this are the mode for the differences which is −47, very close to zero, and the fact that for four horses the difference is positive and for four it is negative.

Thus, the results are consistent with the two formulations delivering the same amount of active agent.

The activity of the product is dependent on the amount of active ingredient that is absorbed by the horse. As the same amount of the active ingredient is absorbed, the pharmacological activity of the two formulations is equivalent.

The lack of a statistically significant difference between the AUC for each formulation means that the products are bioequivalent.

3.1 AUC Results

| Horse Number | AUC for A | AUC for B | Difference |
|---|---|---|---|
| 1 | 9829 | 3486 | 6343 |
| 2 | 2306 | 2023 | 283 |
| 3 | 5689 | 2412 | 3277 |
| 4 | 1613 | 2067 | −454 |
| 5 | 6584 | 8188 | −1604 |
| 6 | 1182 | 1199 | −17 |
| 7 | 966 | 1042 | −76 |
| 8 | 1749 | 1725 | 24 |
| Mean (SD) | 3740 (3246) | 2768 (2317) | |

3.2 Student's t Test
Unpaired t Test

Are the means of AUC A and AUC B equal?

Mean difference=−971.75 (Mean of AUC A minus mean of AUC B)

The 95% confidence interval of the difference: −3996.2 to 2052.7

t=0.6892 with 14 degrees of freedom.

The two-tailed P value is 0.5020, considered not significant.

Test: Are the standard deviations equal?

The t test assumes that the columns come from populations with equal SDs.

The following calculations test that assumption.

F=1.962

The P value is 0.1969.

This test suggests that the difference between the two SDs is not significant.

3.3 Summary of Data

Parameter:

| | AUC A | AUC B |
|---|---|---|
| Mean: | 3739.8 | 2768.0 |
| # of points: | 8 | 8 |
| Std deviation: | 3245.8 | 2317.3 |
| Std error: | 1147.6 | 819.28 |
| Minimum: | 966.00 | 1043.0 |
| Maximum: | 9829.0 | 8188.0 |
| Median: | 2027.5 | 2045.0 |
| Lower 95% CI: | 1025.8 | 830.40 |
| Upper 95% CI: | 6453.7 | 4705.6 |

3.4 Non-Parametric Test

Wilcoxon Signed Ranks Test

Ranks

| | | | | Sum of |
|---|---|---|---|---|
| | | N | Mean Rank | Ranks |
| AUCOLD - AUCNEW | Negative Ranks | 3^{a} | 5.67 | 17.00 |
| | Positive Ranks | 5^{b} | 3.80 | 19.00 |
| | Ties | 0^{c} | | |
| | Total | 8 | | |

^{a}AUCOLD < AUCNEW
^{b}AUCOLD > AUCNEW
^{c}AUCNEW = AUCOLD

Test Statistics^{b}

| | AUCOLD - |
|---|---|
| | AUCNEW |
| Z | −.140^{a} |
| Asymp. Sig. (2-tailed) | .889 |

^{a}Based on negative ranks.
^{b}Wilcoxon Signed Ranks Test
NEW = Test Composition A
OLD = Test Composition B

### Example 7

### Treatment of Inflammation by Intramuscular and Subcutaneous Injections

Methodology

Test Compositions.

For the subcutaneous and intramuscular injections, a composition comprising the [Cu₂(Indo)₄(OH₂)₂] complex in MCT oil was prepared.

The composition comprised the following ingredients:

| Ingredient: | Amount: | |
|---|---|---|
| [Cu₂(Indo)₄(OH₂)₂] complex | 40 | mg |
| Tetraglycol | 300.0 | mg |
| Delios V MCT oil | qs 1.0 | g |

Tetraglycol is the solvent; Delios V MCT oil is a medium chain triglyceride oil.

The composition was prepared as follows:

1 Add tetraglycol to mixer and heat to 75° C. while stirring.

2. Add and dissolve Copper Indomethacin complex. Stir until dissolved, then remove heat.

3. Add Delios V MCT oil, while stirring.

Stir for 15 minutes until homogenous, then allow to cool.

The composition was a single-phase oil and had the appearance of a dark green oil, which is immiscible in water.

The composition contained >95% of Indo in the composition as part of the dimer ([Cu₂(Indo)₄(OH₂)₂] as shown by EPR spectroscopy (Example 4).

For comparison, a similar composition containing IndoH in MCT oil was prepared by the same process using IndoH instead of ([Cu₂(Indo)₄(OH₂)₂].

Animals. Sprague-Dawley rats (weighing 200-250 g) used for these studies were supplied by the laboratory animal services at the University of Sydney. Animals were housed in polypropylene cages and allowed free access to standard laboratory rat chow (Purina Rat Chow, Ralston Purina, St Louis Mo.) and tap water. Animals were housed in the animal care facility of the Faculty of Pharmacy at ambient temperature and humidity with a 12-h light-dark cycle. The experimental animal protocols were approved by the Animal Ethics Committee of the University of Sydney, approval number L07/1-04/3/3846.

In Vivo Anti-inflammatory Activity and Gastric Toxicity. Groups of four rats were used for all studies. All doses were calculated as equivalent concentrations of Indo. Rats were allowed free access to food and water except for gastric toxicity studies, when they were fasted for 24 h but with free access to water. For subcutaneous and intramuscular administration, rats were injected with 125-200 μL volumes of the test compositions (IndoH or [Cu₂(Indo)₄(OH₂)₂] in MCT oil). The control cohort was injected with equivalent volumes of neat MCT. Subcutaneous injections were made in the lower dorsal surface and intramuscular injections were made in the right hind thigh muscle. Inflammation was induced 1 h after dosing by injection of the formulation by an injection of carrageenan (0.1 mL, 1% w/v in isotonic saline) into the plantar region of the hind paw.

Paw volume was measured prior to dosing and at 3 h after carrageenan injection by immersing the left hind paw (to the lateral malleus) into a vessel filled with water as described in Example 5. Immediately after paw volume measurements, 24 h-fasted animals were euthanised and the stomach was excised and opened by incision along the greater curvature. The stomach was rinsed and examined to determine the extent of macroscopic gastric toxicity, which is reported as the summation of the area of macroscopic ulcerations (mm²).

In Vivo Small Intestinal Toxicity. Groups of four rats were used for all studies and were treated similarly as described above, except that they were allowed free access to food and water during the assay. At 24 h after dosing, the entire small intestine was excised and flushed with water to expel the intestinal contents. The intestine was examined from 10 cm distal to the ligament of Treitz to the ileocecal junction, and the toxicity is reported as the summation of the area of macroscopic ulcerations (mm²).

Statistical Analysis. The Student t test was used to compare mean values between two groups and repeated measures ANOVA followed by Bonferroni correction for comparisons was used to compare mean values between more than two groups. Data are expressed as the mean A SEM. All reported P values are two-sided, and P<0.05 was considered statistically significant.

Results

Subcutaneous Injection. The results of dose-response data for the composition containing [Cu₂(Indo)₄(OH₂)₂] in MCT oil are listed in Table 4 together with the data for the composition containing IndoH in MCT at the highest dose at which no small intestinal toxicity was observed for the MCT oil composition containing [Cu₂(Indo)₄(OH₂)₂] in MCT oil. None of the rats exhibited soreness, swelling, redness or any other adverse effect at the site of the injection at all doses.

TABLE 4
Efficacy and Safety of Subcutaneous Treatments of Inflammation
(Rat Paw Oedema) using [Cu₂(Indo)₄(OH₂)₂] or IndoH in MCT oil.

| Dose | | Gastric ulcers | Intestinal ulcers |
|---|---|---|---|
| (mg/kg of Indo) | Percent inhibition | (mm²) | (mm²) |
| 20 | 33.9 | 8, 23, 20, 15 | |
| Fasting | | | |
| 20 | 31.6 | 0, 0, 0, 0 | |
| Non-fasting | | | |
| 20 | | | 70, 80, 110, 20 |
| Non-fasting | | | |
| 10 | 35.8 | 0, 2, 5, 2 | |
| Fasting | | | |
| 10 | | | 2, 3, 0, 1 |
| Non-fasting | | | |
| 7.5 | 47 | | 0, 0, 0, 0 |
| Non-Fasting | | | |
| INDO Alone | 34 | | 3, 5, 6, 4 |
| 7.5 | | | |
| Non-fasting | | | |
| 5 | 24.9 | 0, 1, 0, 0 | |
| Fasting | | | |
| 5 | | | 0, 0, 0, 0 |
| Non-fasting | | | |
| 2 | 6 | 0, 0, 0, 0 | |
| Fasting | | | |
| 2 | | | Not done |
| Non-fasting | | | |
| 1 | 4 | 0, 0, 0, 0 | |
| Fasting | | | |
| 1 | | | Not done |
| Non-fasting | | | |

All doses are given as the amount of Indo delivered in the form of [Cu₂(Indo)₄(OH₂)₂], except for the one example indicated, where Indo was delivered in the form of IndoH.

Although the treatment regimes were not optimized for maximum efficacy, it is clear that this mode of administration has a strong anti-inflammatory effect that plateaus around 5-7.5 mg kg⁻¹ of Indo (the amount of Indo in the complex). At these concentrations, there is no gastrointestinal toxicity induced by the composition containing [Cu₂(Indo)₄(OH₂)₂], either as acute gastric ulcers in fasted rats, or intestinal ulcers due to secondary circulation. By contrast, the composition containing IndoH alone resulted in small intestinal ulceration in all four rats at 7.5 mg/kg and greater ulceration than that observed at 10 mg/kg of Indo for the composition containing [Cu₂(Indo)₄(OH₂)₂]. All gastric side-effects could be easily prevented, even at the very high dose of 20 mg/kg of Indo (administered using the composition containing [Cu₂(Indo)₄(OH₂)₂]) if the rats were not fasted, but at these high concentrations small intestinal ulceration was substantial.

Intramuscular Injection

The results of intramuscular injection into the right hind thigh muscle on rat paw oedema are given in Table 5.

TABLE 5
Efficacy and Safety of Intramuscular Injection Treatments
of Inflammation (Rat Paw Oedema) in Fasting Rats using
[Cu₂(Indo)₄(OH₂)₂] or IndoH in MCT oil.

| | Dose | | Gastric ulcers |
|---|---|---|---|
| Compound | (mg/kg)* | Percent inhibition | (mm²) |
| [Cu₂(Indo)₄(OH₂)₂] | 2 | 0 | 0, 0, 0, 0 |
| IndoH | 5 | 38.7 | 1, 0, 1, 2 |
| [Cu₂(Indo)₄(OH₂)₂] | 10 | 47.7 | 3, 0, 1, 7 |
| IndoH | 10 | 49.4 | 12, 3, 8, 20 |
| [Cu₂(Indo)₄(OH₂)₂] | 20 | 49.7 | 0, 10, 25, 5 |

*Equivalent dose of Indo

While only preliminary data have been obtained, the composition containing [Cu₂(Indo)₄(OH₂)₂] in MCT oil has a similar efficacy and safety profile in rats as those observed following subcutaneous injections, although the efficacy for treatment of inflammation is higher in the plateau region of the dose-response curve. Note that again, while the compositions containing [Cu₂(Indo)₄(OH₂)₂] and IndoH have similar efficacy, the composition containing [Cu₂(Indo)₄(OH₂)₂] in MCT oil resulted in less GI toxicity. At both 5 and 10 mg kg⁻¹ of IndoH, the GI toxicity of the composition containing IndoH in MCT oil was comparable to that observed for twice the dose of Indo when it is delivered as the composition containing [Cu₂(Indo)₄(OH₂)₂] in MCT oil.

Discussion

Both subcutaneous and intramuscular administration of the composition containing the complex [Cu₂(Indo)₄(OH₂)₂] in MCT oil have considerable efficacy, with the latter mode of administration being more efficacious. The results demonstrate the improved safety aspect when the complex is stabilised in a composition of the present invention compared to compositions containing free Indo or IndoH and this opens up treatment regimes that have been restricted with IndoH because of the GI toxicity induced by secondary circulation. If the composition was delivered as a physical mixture of a Cu salt and IndoH or the composition caused the complex to dissociate with the release of free Indo, then toxicity effects similar to those of IndoH are expected.

### Example 8

### Treatment of Inflammation by Topical Creams

Methodology

All rat experiments were performed as outlined in Example 7, except for the following changes.

In Vivo Anti-inflammatory Activity and Gastric Toxicity. For topical administration, rats were treated with test compositions containing [Cu₂(Indo)₄(OH₂)₂] (“CuIndo”), [Zn(Indo)₂(OH₂)₂] (“ZnIndo”), IndoH, or an equivalent Indo and Cu dose of a physical mixture of [Cu₂(OAc)₄(OH₂)₂] and IndoH thoroughly mixed in an emulsifying cream (see below). An amount of 0.2 g of the composition was applied to the right hind paw of each animal and gently massaged in for 1 minute at three-hourly intervals. Inflammation was induced at the final topical application with an injection of carrageenan (0.1 mL, 1% w/v in isotonic saline) into the plantar region of the hind paw.

The composition for topical application was a composition comprising 0.5-2% w/w of Indo delivered as [Cu₂(Indo)₄(OH₂)₂] (“CuIndo”), [Zn(Indo)₂(OH₂)₂] (“ZuIndo”), IndoH or an equivalent Indo and Cu dose of a physical mixture of [Cu₂(OAc)₄(OH₂)₂] and IndoH in an emulsifying cream, the emulsifying cream consisting of:

| | | |
|---|---|---|
| cetomacrogol emulsifying wax | 15 | g |
| liquid paraffin | 10 | g |
| white soft paraffin | 10 | g |
| chlorocresol | 0.1 | g |
| propylene glycol | 5 | ml |
| purified and cooled water | to 100 | g. |

TABLE 6
Comparison of Efficacy (Treatment of Rat Paw Oedema) and Safety (Small
Intestinal Toxicity) of Equivalent Indo Doses (0.5-2% of Indo) of Topical
Formulations of [Cu₂(Indo)₄(OH₂)₂], [Zn(Indo)₂(OH₂)₂], IndoH, or a physical mixture
of [Cu₂(OAc)₄(OH₂)₂] and IndoH to the Paws of Rats (Using Emulsifying Cream as
the Vehicle)

| | IndoH | | CuIndo | | ZnIndo | |
|---|---|---|---|---|---|---|
| Conc. | Efficacy | S I ulcers^{a} | Efficacy | S I ulcers^{a} | Efficacy | S I ulcers^{a} |
| (% equiv) | (% inhib) | (mm²) | (% inhib) | (mm²) | (% inhib) | (mm²) |
| 2 | 42 | 45, 35, 30, 85 | 55.2 | 3, 5, 40, 45 | | |
| 1 | 35 | 26, 8, 110,^{b} 34 | 52 | 6, 3, 0, 0 | 41 | 10, 15, 4, 0 |
| | 58^{c} | 60, 180, 35, 45^{c} | | | | |
| 0.75 | 60.8 | 0, 0, 0, 0 | 73 | 0, 0, 0, 0 | | |
| 0.5 | 18.6 | 0, 0, 0, 0 | 12.2 | 0, 0, 0, 0 | 29 | 0, 0, 0, 0 |

^{a}Small intestinal ulceration.
^{b}Overt intestinal bleeding.
^{c}A physical mixture of [Cu₂(OAc)₄(OH₂)₂] and IndoH.

Table 6 shows that the topical formulation containing [Cu₂(Indo)₄(OH₂)₂] was both far safer and more efficacious, with substantial small intestinal ulceration only observed in 2% formulations whereas even in 1% formulations with IndoH small intestinal ulceration is substantial and one rat even had obvious intestinal bleeding. The formulation containing [Zn(Indo)₂(OH₂)₂] also results in higher efficacy and decreased GI toxicity compared with the composition containing IndoH. It is somewhat less efficacious and more toxic than the Cu complex in 1% equivalent preparations, but is superior to both the composition containing IndoH and the composition containing [Cu₂(Indo)₄(OH₂)₂] in terms of efficacy at the low dose of 0.5%. While a composition containing a physical mixture of [Cu₂(OAc)₄(OH₂)₂] and IndoH has similar efficacy as the composition containing [Cu₂(Indo)₄(OH₂)₂], when both compositions contained the same amount of Indo (1% w/w) and Cu, the composition containing the mixture was even more GI toxic than IndoH alone. The data support strongly the hypothesis that the Cu and Zn complexes remain intact in the cream until they are absorbed, since a physical mixture of IndoH and CuAcetate is even more GI toxic than IndoH alone.

Discussion.

Topical applications of IndoH are rare because of the severe small intestinal toxicity that arises from topical applications that are therapeutically active. This problem is also exemplified in the present example where there is a very narrow therapeutic window between the onset of life-threatening internal bleeding and a rapid drop-off in efficacy for the topical application of the composition containing IndoH. By contrast, the compositions containing Cu-Indo or Zn-Indo have far superior therapeutic windows, higher efficacy and much lower toxicity at equivalent therapeutic doses. They are also considerably less GI toxic than equivalent compositions containing a physical mixture of [Cu₂(OAc)₄(OH₂)₂] and IndoH. These results demonstrate the importance of topical formulations of the present invention for solubilising and stabilizing the complexes in colloidal emulsions or compositions that are immiscible in water in order for the safe and efficacious delivery of the drugs for veterinary and human applications.

It should also be noted that the therapeutic window will be enhanced for larger animals, such as dogs or horses, or for humans, since the same application on a given area of skin, will result in much lower doses (in mg kg⁻¹) for the animal or human, as the weight increases, hence GI toxicity considerably reduced when such applications are used for dogs, and more particularly, humans and horses.

### Example 9

### Efficacy and Safety of Ophthalmic Formulations

Indomethacin can cause adverse ocular effects (such as corneal deposits and retinal disturbances), but the delivery of indomethacin as the complex has the potential to provide a much safer delivery mode.

Various compositions of the present invention for ophthalmic administration can be prepared, and three are exemplified below.

Composition 1 (“Cu-Algesic eye drops”) consisting of (1% w/v [Cu₂(Indo)₄(OH₂)₂] in an aqueous micelle of polyvinyl alcohol (14 mg mL⁻¹) and povidone (14 mg mL⁻¹).

Composition 2 (“Cu-Algesic eye ointment”) consisting of (1% w/w [Cu₂(Indo)₄(OH₂)₂] in lacrilube (white soft paraffin 57.3%, mineral oil (liquid paraffin) 42.5%, lanolin alcohols 0.2%) containing 1,1,1-trichloro-2-methyl-2-propanol (0.5%). This composition is immiscible in water. The ratio of white soft paraffin and liquid paraffin can be changed in order to provide an ointment with different degrees of thickness depending on the application.

Composition 3 consisting of [Cu₂(Indo)₄(OH₂)₂] (0.125% w/w), tetraglycol (0.875% w/w), Cremophore EL (1% w/w), MCT oil (48% w/w) and White Parrafin Jelly BP (50% w/w). This composition is immiscible in water.

As an example of the efficacy and safety of the opthalmological application of compositions of the present invention, 0.5 mL of Composition 3 was applied topically to the eyes of 10 horses and no adverse effects were observed.

Composition 3 was also used clinically as described below.

A foal born with severe conjunctivitis, was treated with a conventional ophthalmic antibiotic ointment and the condition progressed to hypophyon (pus in anterior chamber, eye had white appearance). 0.5 mL of the eye ointment of Composition 3 was topically applied to the eyes once daily, and the eye returned to normal by 10 days.

0.5 mL of the eye ointment of Composition 3 was also topically applied once daily to the eyes of two horses with uveitis (inflammation of uvea within eye). The signs of this condition are usually a swollen and painful eye, In both cases, the inflammation cleared after 7 days of treatment with Composition 3.

No adverse effects from the treatment were observed in any of the horses treated, with vision apparently having returned to normal.

The results shown above demonstrate that the ointments can be used safely and there is preliminary evidence that they are very effective in the treatment of severe ophthalmic conditions, which, in the case of the foal, was untreatable by conventional therapies and would have resulted in the foal having to be euthanised.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

1. A pharmaceutical composition comprising a metal complex of a carboxylate having anti-inflammatory activity in a pharmaceutically acceptable carrier, wherein:
(1) the composition has a colloidal structure, or forms a colloidal structure when administered to a human or animal, or is immiscible with water;
(2) more than 80% of the total amount of the carboxylate having anti-inflammatory activity in the composition is present as part of a metal complex; and
(3) less than 10% of the carboxylate having anti-inflammatory activity complexed with the metal dissociates from the metal over 12 months when the composition is stored in the absence of light at room temperature;
but excluding compositions comprising a metal complex containing the ligand DMF.

2. A pharmaceutical composition according to claim 1, wherein less than 10% of the carboxylate having anti-inflammatory activity complexed with the metal dissociates from the metal over 18 months when the composition is stored in the absence of light at room temperature.

3. A pharmaceutical composition according to claim 1, wherein less than 5% of the carboxylate having anti-inflammatory activity complexed with the metal dissociates from the metal over 18 months when the composition is stored in the absence of light at room temperature.

4. A pharmaceutical composition according to claim 1, wherein the carboxylate having anti-inflammatory activity is a NSAID.

5. A pharmaceutical composition according to claim 1, wherein the metal is Cu, Zn, Co or Ni.

6. A pharmaceutical composition according to claim 1, wherein the metal complex of a carboxylate having anti-inflammatory activity is selected from the group consisting of:
[Cu₂(Sup)₄(OH₂)₂],
[Cu(Tol)₂(pyridine)₂],
[Cu₂(Tol)₄(dmso)₂],
[Cu(Nap)₂(pyridine)₂],
[Cu₂(Nap)₄(dmso)₂],
[Cu(lbu)₂(pyridine)₂],
[Cu₂(lbu)₄(dmso)₄],
[Cu(lbu)₂(imidazole)₂],
[Cu(lbu)₂(2-methylimidazole)₂],
[Cu₂(lbu)₄(caffeine)₂],
[Cu₂(lbu)₄(metronidazole)₂],
[Cu₂(Flufen)₄L₂] where each L is independently selected and is caffeine or papaverine,
[Cu(Flufen)₂L₂] where each L is independently selected and is nicotine, nicotinamide or N,N-diethylnicotinamide,
[Cu(Nif)₂L₂] where each L is independently selected and is 3-pyridylmethanol or water,
[Cu₂(Nif)₄(dmso)₂],
[Cu₂(Indo)₄L₂] where each L is independently selected and is water, N,N-dimethylacetamide, N-methyl-2-pyrrolidone, tetrahydrofuran, acetonitrile, acetone or dimethylsulfoxide,
[Cu₂(Dic)₄L₂] wherein each L is independently selected and is water, ethanol, dimethylsulfoxide or methanol,
and mixtures thereof.

7. A pharmaceutical composition according to claim 1, wherein the carboxylate having anti-inflammatory activity is indomethacin.

8. A pharmaceutical composition according to claim 7, wherein the metal complex of a carboxylate having anti-inflammatory activity is a dinuclear metal complex containing indomethacin.

9. A pharmaceutical composition according to claim 8, wherein the metal complex of a carboxylate having anti-inflammatory activity is [Cu₂(Indo)₄(OH₂)₂].

10. A pharmaceutical composition according to claim 1, wherein the metal complex of a carboxylate having anti-inflammatory activity is a mononuclear copper complex of the formula (1):
[Cu(η²-L¹)₂L₂]^{p} (1)
wherein “η²-L¹” is a bidentate ligand of the formula L¹: 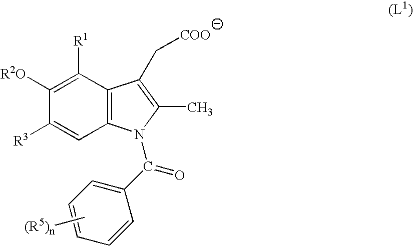
wherein:
R¹ is H or halo;
R² is H; a C₁ to C₆ alkyl, an alkenyl or an alkynyl, where the C₁ to C₆ alkyl, alkenyl or alkynyl may be optionally substituted; or 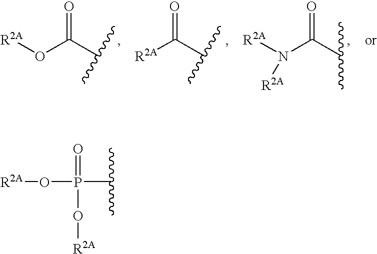
wherein each R^{2A} is independently selected from the group consisting of H, C₁ to C₆ alkyl, alkenyl, alkynyl, aryl, cycloalkyl and arylalkyl, where the C₁ to C₆ alkyl, alkenyl, alkynyl, aryl, cycloalkyl or arylalkyl may be optionally substituted;
R³ is H or halo;
each R⁵ is independently selected from the group consisting of halo, —CH₃, —CN, —OCH₃, —SCH₃ and —CH₂CH₃, where the —CH₃, —OCH₃, —SCH₃ or —CH₂CH₃ may be optionally substituted; and
n is 1, 2, 3, 4 or 5;
each L is independently selected and is a monodentate ligand,
and p is the charge of the complex.

11. A pharmaceutical composition according to claim 10, wherein L¹ is Indo.

12. A pharmaceutical composition according to claim 1, wherein the composition has a colloidal structure selected from micelles in an aqueous carrier or an oil-in-water emulsion.

13. A pharmaceutical composition according to claim 1, wherein the composition forms a colloidal structure when administered to a human or animal.

14. A pharmaceutical composition comprising a metal complex of a carboxylate having anti-inflammatory activity, one or more pharmaceutically acceptable surfactants and water, wherein:
(1) the composition comprises micelles in an aqueous carrier;
(2) more than 80% of the total amount of the carboxylate having anti-inflammatory activity in the composition is present as part of a metal complex; and
(3) less than 10% of the carboxylate having anti-inflammatory activity complexed with the metal dissociates from the metal over 12 months when the composition is stored in the absence of light at room temperature;
but excluding compositions comprising a metal complex containing the ligand DMF.

15. A pharmaceutical composition according to claim 14, wherein the one or more surfactants is selected from the group consisting of Sorbitan Fatty Acid Esters surfactants and Caster Oil Polyoxyethylene surfactants.

16. A pharmaceutical composition comprising a metal complex of a carboxylate having anti-inflammatory activity, one or more pharmaceutically acceptable oils, one or more pharmaceutically acceptable surfactants and water, wherein:
(1) the composition is an oil-in-water emulsion;
(2) more than 80% of the total amount of the carboxylate having anti-inflammatory activity in the composition is present as part of a metal complex; and
(3) less than 10% of the carboxylate having anti-inflammatory activity complexed with the metal dissociates from the metal over 12 months when the composition is stored in the absence of light at room temperature;
but excluding compositions comprising a metal complex containing the ligand DMF.

17. A pharmaceutical composition according to claim 16, wherein the one or more oils is a medium chain triglyceride.

18. A pharmaceutical composition according to claim 16, wherein the one or more surfactants is selected from the group consisting of Sorbitan Fatty Acid Esters surfactants and Caster Oil Polyoxyethylene surfactants.

19. A pharmaceutical composition comprising a metal complex of a carboxylate having anti-inflammatory activity in a pharmaceutically acceptable carrier, wherein:
(1) the composition is immiscible with water;
(2) more than 80% of the total amount of the carboxylate having anti-inflammatory activity in the composition is present as part of a metal complex; and
(3) less than 10% of the carboxylate having anti-inflammatory activity complexed with the metal dissociates from the metal over 12 months when the composition is stored in the absence of light at room temperature.

20. A pharmaceutical composition for oral administration comprising a metal complex of a carboxylate having anti-inflammatory activity, one or more pharmaceutically acceptable oils and one or more pharmaceutically acceptable surfactants, wherein:
(1) the one or more oils and one or more surfactants are present in the composition in amounts such that following oral administration of the composition to a human or animal, the composition forms an oil-in-water emulsion on contact with aqueous fluids in the digestive system of the human or animal;
(2) more than 80% of the total amount of the carboxylate having anti-inflammatory activity in the composition is present as part of a metal complex; and
(3) less than 10% of the carboxylate having anti-inflammatory activity complexed with the metal dissociates from the metal over 12 months when the composition is stored in the absence of light at room temperature;
but excluding compositions comprising a complex containing the ligand DMF.

21. A pharmaceutical composition according to claim 20, wherein the one or more oils is a medium chain triglyceride.

22. A pharmaceutical composition according to claim 20 or 21, wherein the one or more surfactants is selected from the group consisting of Sorbitan Fatty Acid Esters surfactants and Caster Oil Polyoxyethylene surfactants.

23. A pharmaceutical composition comprising:

| | Amount (% by weight |
|---|---|
| Ingredient: | of the composition): |
| One or more metal complexes of a carboxylate | 3 to 7 |
| having anti-inflammatory activity | |
| One or more solvents | 20 to 40 |
| One or more surfactants | 5 to 20 |
| One or more thickeners | 0 to 15 |
| Medium chain triglyceride | 40 to 60 |

24. A pharmaceutical composition according to claim 23 comprising:

| | Amount (% by weight |
|---|---|
| Ingredient: | of the composition): |
| One or more metal complexes of a carboxylate | 3 to 7 |
| having anti-inflammatory activity | |
| One or more solvents | 30 ± 10% |
| One or more surfactants | 10 ± 10% |
| One or more thickeners | 5 ± 10% |
| Medium chain triglyceride | 50 ± 10% |

25. A pharmaceutical composition according to claim 13, wherein the carboxylate having anti-inflammatory activity is a NSAID.

26. A pharmaceutical composition according to claim 13, wherein the carboxylate having anti-inflammatory activity is indomethacin.

27. A method for treating an inflammatory condition in a human or animal, the method comprising administering to the human or animal a therapeutically effective amount of a composition according to claim 1, 14, 16, 19, 20, 23 or 24.

28. A method according to claim 27, wherein the inflammatory condition is selected from the group consisting of rheumatoid arthritis, osteoarthritis, acute musculoskeletal disorders, lower back pain, and inflammation, pain or edema following a surgical or non-surgical procedure.

29. A method according to claim 27, wherein the inflammatory condition is psoriasis or psoriatic arthritis.
